(19) United States
(12) Patent Application Publication
(10) Pub. No.: US 2025/0388883 A1
(43) Pub. Date: Dec. 25, 2025

(54) GENE EDITING COMPOSITIONS AND METHODS OF USE THEREOF

(71) Applicant: Demeetra AgBio, Inc., Lexington, KY (US)

(72) Inventors: John CRAWFORD, Lexington, KY (US); Corey BRIZZEE, Lexington, KY (US)

(21) Appl. No.: 18/877,648

(22) PCT Filed: Jun. 30, 2023
(86) PCT No.: PCT/US2023/069536

§ 371 Date: Dec. 20, 2024

Related U.S. Application Data
(60) Provisional application No. 63/357,588, filed on Jun. 30, 2022.

(51) Int. Cl. C12N 9/22; C12N 15/11; C12N 15/82; C12N 15/90

(52) U.S. Cl. CPC C12N 9/22; C12N 9/226; C12N 15/11; C12N 15/8213; C12N 15/907; C07K 2319/00; C12N 2310/20; C12N 2800/80

(57) ABSTRACT

The disclosure provides improved Cas-CLOVER systems for gene editing. In embodiments, the disclosure provides recombinant Clo051 endonucleases, or nuclease domains thereof, comprising one or more amino acid mutations (e.g. one or more amino acid substitutions at E101 and/or F44). The disclosure also provides fusion proteins, comprising: a DNA localization component, and any one of the Clo051 endonucleases disclosed herein or the nuclease domains thereof, and further provides methods of using the fusions proteins in gene editing, including introducing a double stranded break in a target nucleic acid.

### CROSS REFERENCE TO RELATED APPLICATIONS

The present Application claims the benefit of priority to U.S. Provisional Application No. 63/357,588, filed on Jun. 30, 2022, the contents of which are hereby incorporated by reference in their entirety.

### REFERENCE TO AN ELECTRONIC SEQUENCE LISTING

The contents of sequence listing the electronic (DMAI_005_01WO_SeqList_ST26.xml; Size: 419,398 bytes; and Date of Creation: Jun. 30, 2023) are herein incorporated by reference in its entirety.

### BACKGROUND

The Cas-CLOVER is a targeted gene editing system that is more precise than conventional CRISPR-Cas systems. The Cas-CLOVER system uses a fusion protein, comprising a Clo051 Type II endonuclease and a nuclease-inactivated Cas protein, in combination with a pair of guide RNAs (gRNAs) to catalyze a double stranded break in a target nucleic acid. The Cas-CLOVER system is highly stringent and has low off-target activity because it's activity is promoted by the dimerization of the Clo051 endonuclease or a nuclease domain thereof and the binding of both gRNAs to their respective target regions.

However, there is an unmet need to improve the efficiency of the Cas-CLOVER system, and reduce its cellular toxicity, which are discussed further in this disclosure.

### SUMMARY

The disclosure provides recombinant Clo051 endonucleases, or nuclease domains thereof, wherein the Clo051 endonuclease or a nuclease domain thereof comprises an amino substitution at E101, F44, or a combination thereof; and fusion proteins comprising: (i) a DNA localization component, and (ii) any one of the Clo051 endonucleases or the nuclease domains thereof disclosed herein. In embodiments, the DNA localization component comprises a catalytically inactive Cas protein, or a DNA binding domain thereof. In embodiments, the catalytically inactive Cas9 (dCas9) lacks a C-terminal SV40 nuclear localization sequence (NLS).

The disclosure further provides compositions, comprising: (a) a left guide RNA (gRNA) and a right gRNA; and (b) any one of the fusion proteins disclosed herein. In embodiments, the 5′ end of the left gRNA and/or the 5′ end of the right gRNA are conjugated to a tRNA linker. In embodiments, the composition is capable of catalyzing a double stranded break in the target nucleic acid.

The disclosure also provides methods of introducing a double stranded break in a target nucleic acid, the method comprising: bringing any one of the compositions disclosed herein in contact with the target nucleic acid. In embodiments, the cutting efficiency of the composition is higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof. In embodiments, the cellular toxicity of the composition is lower than, or the same as, a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof.

The disclosure provides methods of modifying a target double stranded nucleic acid, comprising: bringing (a) any one of the compositions disclosed herein and (b) a donor nucleic acid, in contact with the target nucleic acid, wherein the donor nucleic acid is capable of homologous recombination with the target nucleic acid.

These and other embodiments are addressed in more detail in the detailed description set forth below.

### BRIEF DESCRIPTION OF THE FIGURES

FIG. 1A is a schematic representation of the Cas-CLOVER gene editing system. The Cas9-Clo051 fusion protein dimerizes when it is recruited by the left gRNA and the right gRNA to the target site leading to the induction of a double stranded break by Clo051 or a nuclease domain thereof between the left protospacer adjacent motif (PAM) sequence and the right PAM sequence. FIG. 1B is a schematic that depicts the homologous recombination underlying the ADE2 reporter assay. Cas-CLOVER-mediated induction of a double stranded break in the ADE2 gene results in the homologous recombination of a donor DNA at the target ADE2 gene site, resulting in the deletion of the ADE2 gene and the red colony phenotype, as described in Example 1.

FIG. 2A is a bar graph showing the total number of colonies (on the left Y axis) and a scatter plot of the % cutting efficiency (on the right Y axis) obtained upon expression of a mutant version of the dCas9-Clo051 fusion protein, comprising a mutant Clo051 nuclease domain comprising either one of the amino acid substitutions: F44S, F44T or F44A, or the control dCas9-Clo051 protein (108.1 WT) in yeast cells under the control of the high strength promoter, ADH1, together with a left gRNA and a right gRNA targeting the ADE2 gene. FIG. 2B is a bar graph showing the total number of colonies (on the left Y axis) and a scatter plot of the % cutting efficiency (on the right Y axis) obtained upon expression of a mutant version of the dCas9-Clo051 fusion protein, wherein Clo051 nuclease domain comprises either one of the amino acid substitutions: F44S, F44T or F44A, or the control dCas9-Clo051 protein comprising a nuclease domain of Clo051 having amino acid sequence of SEQ ID NO: 23 (108.4 WT) in yeast cells under the control of the low strength REVI promoter, together with a left gRNA and a right gRNA targeting the ADE2 gene.

FIG. 3 is a schematic representation of a monomer of Clo051 that was aligned to FokI dimer structure bound to DNA.

FIG. 4 is a bar graph showing the total number of colonies (on the left Y axis) and a scatter plot of the % cutting efficiency (on the right Y axis) obtained upon expression of a mutant version of the dCas9-Clo051 fusion protein, comprising a mutant nuclease domain of Clo051 comprising either one of the amino acid substitutions: E101S, E101N, or E101A or the control dCas9-Clo051 protein comprising a nuclease domain of Clo051 having amino acid sequence of SEQ ID NO: 23 (108.4 WT) in yeast cells under the control of the low strength REV1 promoter, together with a left gRNA and a right gRNA targeting the ADE2 gene.

FIG. 5 is a bar graph showing the total number of colonies (on the left Y axis) and a scatter plot of the % cutting efficiency (on the right Y axis) obtained upon expression of a mutant version of the dCas9-Clo051 fusion protein, comprising a mutant nuclease domain of Clo051 comprising either one of the amino acid substitutions: F44S, F44T, F44A, E101S, E101N, or E101A or the control dCas9-Clo051 protein comprising a nuclease domain of Clo051 having amino acid sequence of SEQ ID NO: 23 (108.4 WT) in yeast cells under the control of the low strength promoter, REV1, together with a left gRNA and a right gRNA targeting the ADE2 gene. Additionally, “dC” indicates that the C-terminal SV40 nuclear localization (NLS) sequence of the dCas9 is deleted in that fusion protein, and “5′ linker” indicates that each of the 5′ end of the left gRNA and the 5′ end of the right gRNA are conjugated to a tRNA linker, comprising a nucleic acid sequence of SEQ ID NO: 111. See Example 4 for more details.

FIG. 6 shows a bar graph showing the total number of colonies (on the left Y axis) and a scatter plot of the % cutting efficiency (on the right Y axis) obtained upon expression of a mutant version of the dCas9-Clo051 fusion protein, comprising a mutant nuclease domain of Clo051 comprising either one of the amino acid substitutions: E101S, E101N, E101A, E101L, E101I, E101G, E101T, E101F, E101Y, E101W, E101P, E101H, E101Q, E101R, E101M, E101K, E101V, E101D, E101C, or the control dCas9-Clo051 protein comprising a nuclease domain of Clo051 having amino acid sequence of SEQ ID NO: 23 (108.4 WT) in yeast cells under the control of the low strength promoter, REV1, together with a left gRNA and a right gRNA targeting the ADE2 gene. Additionally, on the X-axis, “dC” indicates that the C-terminal SV40 nuclear localization (NLS) sequence of the dCas9 is deleted in that fusion protein, and “5′ linker” indicates that each of the 5′ end of the left gRNA and the 5′ end of the right gRNA are conjugated to a tRNA linker, comprising a nucleic acid sequence of SEQ ID NO: 111. See Example 4 for more details.

FIG. 7 shows a bar graph showing the total number of colonies (on the left Y axis) obtained upon expression of different Cas-CLOVER systems as explained in Example 5. “dC” indicates that the C-terminal SV40 nuclear localization (NLS) sequence of the dCas9 is deleted in that fusion protein, and “5′ linker” indicates that each of the 5′ end of the left gRNA and the 5′ end of the right gRNA are conjugated to a tRNA linker, comprising a nucleic acid sequence of SEQ ID NO: 111.

FIG. 8 shows a bar graph showing the % cutting efficiency (on the right Y axis) obtained upon expression of different Cas-CLOVER systems as explained in Example 6. “dC” indicates that the C-terminal SV40 nuclear localization (NLS) sequence of the dCas9 is deleted in that fusion protein, and “5′ linker” indicates that each of the 5′ end of the left gRNA and the 5′ end of the right gRNA are conjugated to a tRNA linker, comprising a nucleic acid sequence of SEQ ID NO: 111.

### DETAILED DESCRIPTION

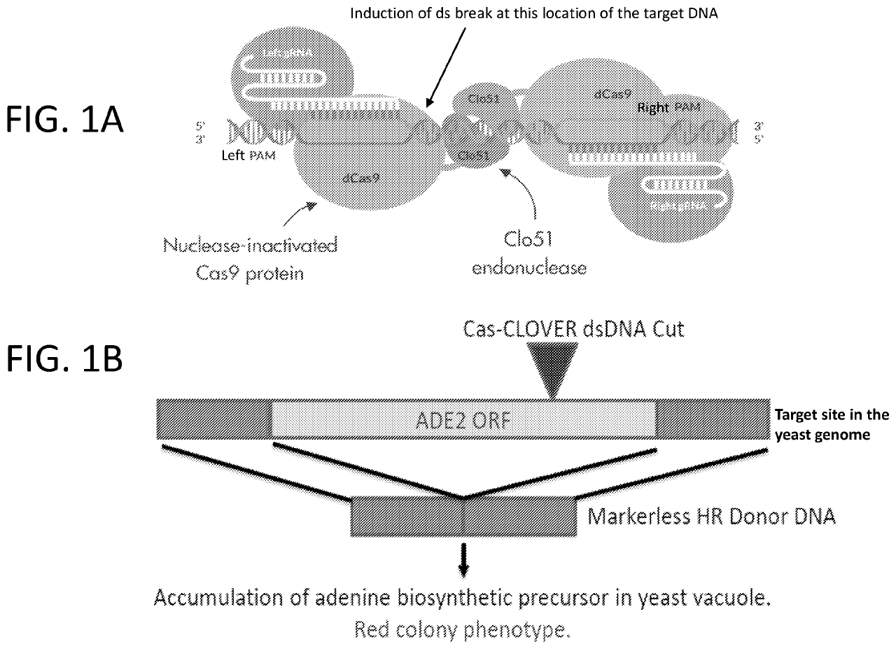

The Cas-CLOVER gene editing system uses a fusion protein comprising a catalytically inactive Cas protein (e.g. dCas9) and a Clo051 endonuclease, or a nuclease domain thereof to catalyze the formation of a double stranded break in a target nucleic acid resulting in homologous recombination of a donor nucleic acid at the target site (FIG. 1A). Further details of the Cas-CLOVER system are provided in U.S. Patent Publication US2018/0187185, which is incorporated herein by reference in its entirety for all purposes. A disadvantage of the Cas-CLOVER system is that its cutting efficiency can be low (sometimes less than 50%). Moreover, the expression of dCas9-Clo051, particularly from strong promoters, can be toxic to cells.

The disclosure provides improved Cas-CLOVER gene editing systems having enhanced cutting efficiency and lower cellular toxicity. In embodiments, the improved Cas-CLOVER gene editing systems disclosed herein utilize dCas9-Clo051 fusion proteins, comprising a Clo051 endonuclease, or a nuclease domain thereof that has an amino acid substitution at the amino acid residues F44 and/or E101. Furthermore, in embodiments, the improved Cas-CLOVER gene editing systems disclosed herein utilize dCas9-Clo051 fusion proteins that comprise a version of dCas9 that lacks a C-terminal SV40 nuclear localization signal (NLS). Also, in embodiments, the improved Cas-CLOVER gene editing systems disclosed herein utilize a pair of gRNAs that are each conjugated to a tRNA linker at their 5′ end.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

As used herein, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, the term “wild type” refers to a typical form of an organism, strain, gene, protein, or characteristic as it occurs in nature as distinguished from mutant or variant forms. For example, a wild type protein is the typical form of that protein as it occurs in nature.

The term “mutant protein” is a term of the art and refers to a protein that is distinguished from the wild type form of the protein on the basis of the presence of one or more amino acid modifications, such as, for example, one or more amino acid substitutions, insertions, deletions, or a combination thereof. The term “mutant gene” is a term of the art and refers to a gene that is distinguished from the wild type form of the gene on the basis of the presence of one or more nucleic acid modifications, such as, for example, one or more nucleic acid substitutions, insertions, deletions, or a combination thereof. In embodiments, a mutant gene encodes a mutant protein.

An amino acid modification may be an amino acid substitution, amino acid deletion and/or amino acid insertion. An amino acid substitution may be a conservative amino acid substitution or a non-conservative amino acid substitution. An amino acid substitution at a specific position on the protein sequence is denoted herein in the following manner: “one letter code of the WT amino acid residue-amino acid position-one letter code of the amino acid residue that replaces this WT residue”. For example, a mutant version of a Clo051 which has an amino acid substitution of E101S refers to a Clo051 protein in which the wild type residue at the 101^{st} position (E or glutamic acid) is replaced with S or serine.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539.

The term “subject” refers to a vertebrate or invertebrate, such as a mammal or a plant, fungi or bacteria. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In embodiments, the adults are seniors about 65 years or older, or about 60 years or older. In embodiments, the subject is a pregnant woman or a woman intending to become pregnant. The plant may be a monocot or dicot such as corn, soy bean, wheat, rice, cotton, canola, banana, tobacco, cannabis, tomato, potato, lettuce or green bean. The fungi may be yeast or mushrooms or filamentous fungi. The bacteria is not limited and may be Escherichia Coli, Pseudomonas spp. or any bacteria commonly used in protein manufacturing.

The term, “guide nucleic acid,” as used herein refers to a nucleic acid comprising: a first nucleotide sequence that hybridizes to a target nucleic acid; and a second nucleotide sequence that is capable of being non-covalently bound by an effector protein, such as, dCas9. The Cas-CLOVER systems disclosed herein employ two gRNAs—a “left guide RNA” that binds upstream of the double strand break target site, and a “right guide RNA” that binds downstream of the double strand break target site, as shown in FIG. 1A.

The term, “effector protein,” as used herein refers to a protein, polypeptide, or peptide that non-covalently binds to a guide nucleic acid (e.g. a guide RNA or gRNA) to form a complex that contacts a target nucleic acid, wherein at least a portion of the guide nucleic acid hybridizes to a target sequence of the target nucleic acid (e.g. Cas9). In embodiments, the effector protein does not modify the target nucleic acid, but it is fused to a fusion partner protein that modifies the target nucleic acid (e.g. Clo051-dCas9 fusion proteins disclosed herein). A non-limiting example of modifying a target nucleic acid is cleaving (hydrolysis) of a phosphodiester bond.

“dCas” as used herein refers to an effector protein that is modified relative to a naturally-occurring effector protein to have a reduced or eliminated catalytic activity relative to that of the naturally-occurring effector protein, but retains its ability to interact with a guide nucleic acid. For example, “dCas9” refers to a variant of the Cas9 protein that is modified relative to the naturally-occurring Cas9 to have a reduced or eliminated catalytic activity relative to that of naturally-occurring Cas9, but retains its ability to interact with a guide nucleic acid; “dCas2” refers to a variant of the Cas2 protein that is modified relative to the naturally-occurring Cas2 to have a reduced or eliminated catalytic activity relative to that of naturally-occurring Cas2, but retains its ability to interact with a guide nucleic acid, and so on. In embodiments, dCas proteins contain domains or sequences from multiple species of bacteria and other organisms.

The catalytic activity that is reduced or eliminated is often a nuclease activity. The naturally-occurring effector protein may be a wildtype protein. In embodiments, the dCas protein is referred to as a catalytically inactive variant of an effector protein, e.g., a Cas effector protein. In embodiments, the dCas protein is an engineered Cas protein comprising a mutation in a nuclease domain relative to the corresponding wildtype Cas protein, wherein the engineered Cas protein provides reduced nuclease activity relative to the wildtype Cas protein, as measured by a nucleic acid cleavage assay.

The term, “donor nucleic acid,” as used herein refers to a nucleic acid that is incorporated into a target nucleic acid.

As used herein, “cutting efficiency” relates to a measure of the effectiveness of the Cas-CLOVER system in generating double stranded breaks in a target nucleic acid. “Cutting efficiency” may be calculated by measuring the abundance of double stranded breaks generated in a target nucleic acid molecule in a sample, normalized to the abundance of the Cas-CLOVER system in the sample, and the abundance of the target nucleic acid molecule in the sample. In embodiments, the cutting efficiency, expressed as a percentage, is obtained using the ADE2 reporter assay described herein.

### Recombinant Clo051 Endonucleases, and Nuclease Domains Thereof

The disclosure provides recombinant Clo051 endonucleases, or nuclease domains thereof, comprising one or more amino acid mutations (e.g. one or more amino acid substitutions, one or more amino acid insertions, and/or one or more amino acid deletions).

In embodiments, the wild type Clo051 endonuclease is the NCBI Reference Sequence: WP_008676092.1, derived from the genome of Clostridium spec. 7_2_43FAA. In embodiments, the wild type Clo051 endonuclease comprises the amino acid sequence of SEQ ID NO: 117. Further details on Clo051 endonuclease are provided in WO2012168304A1, which is incorporated by reference in its entirety for all purposes.

In embodiments, the nuclease domain of Clo051 endonuclease comprises amino acid residues 389 to 587 of SEQ ID NO: 117. In embodiments, the nuclease domain of the Clo051 endonuclease comprises the amino acid sequence of SEQ ID NO. 71.

In embodiments, the nuclease domain of Clo051 endonuclease comprises amino acid residues 389 to 587 of SEQ ID NO: 117 and an N-terminal SV40 nuclear localization signal (NLS; SEQ ID NO: 116). In embodiments, the nuclease domain of the Clo051 endonuclease comprises the amino acid sequence of SEQ ID NO. 118.

In embodiments, the nuclease domain of Clo051 endonuclease comprises amino acid residues 389 to 587 of SEQ ID NO: 117, an N-terminal SV40 nuclear localization signal (NLS) and a ‘GS’ linker between the NLS and the nuclease domain. In embodiments, the nuclease domain of the Clo051 endonuclease comprises the amino acid sequence of SEQ ID NO. 23.

In embodiments, the Clo051 endonuclease, or a nuclease domain thereof comprises one or more linkers comprising the amino acid sequence of any one or more of the following: SEQ ID Nos. 119-140. In embodiments, the Clo051 endonuclease, or a nuclease domain thereof comprises one or more linkers comprising the amino acid sequence of any one or more of the following: SEQ ID Nos. 119-140 between the NLS and the nuclease domain.

The disclosure provides recombinant Clo051 endonucleases, or nuclease domains thereof, comprising: (i) an amino substitution at E101 of SEQ ID NO: 23, or at a corresponding amino acid residue, of a wild type Clo051 endonuclease or a nuclease domain thereof, (ii) an amino substitution at F44 of SEQ ID NO: 23, or at a corresponding amino acid residue, of a wild type Clo051 endonuclease or a nuclease domain thereof, or (iii) a combination thereof.

In embodiments, the recombinant Clo051 endonuclease, or the nuclease domain thereof comprises up to 10 amino acid substitutions relative to SEQ ID NO: 23 or the amino acid sequence of a wild type Clo051 endonuclease or a nuclease domain thereof. In embodiments, the recombinant Clo051 endonuclease, or the nuclease domain thereof comprises: an amino substitution at E90, F33, or a combination thereof relative to the amino acid sequence of SEQ ID NO: 71. In embodiments, the recombinant Clo051 endonuclease, or the nuclease domain thereof comprises: an amino substitution at E101, F44, or a combination thereof relative to the amino acid sequence of SEQ ID NO: 23. In embodiments, the recombinant Clo051 endonuclease, or the nuclease domain thereof comprises: an amino substitution at E478, F421, or a combination thereof relative to the amino acid sequence of SEQ ID NO: 117. In embodiments, the recombinant Clo051 endonuclease, or the nuclease domain thereof comprises: an amino substitution at E99, F42, or a combination thereof relative to the amino acid sequence of SEQ ID NO: 118.

The disclosure provides recombinant Clo051 endonucleases, or a nuclease domains thereof, comprising: (i) an amino substitution at E101 of SEQ ID NO: 23, or at the corresponding amino acid residue of SEQ ID NO: 71, SEQ ID NO: 117 or SEQ ID NO: 118, (ii) an amino substitution at F44 of SEQ ID NO: 23, or at the corresponding amino acid residue of SEQ ID NO: 71, SEQ ID NO: 117 or SEQ ID NO: 118, or (iii) a combination thereof. In embodiments, the recombinant Clo051 endonuclease, or the nuclease domain thereof comprises up to 10 (for example, 1, 2, 3, 4, 5, 6, 7, 8, 9, or 10) amino acid substitutions relative to SEQ ID NO: 23, SEQ ID NO: 71, SEQ ID NO: 117 or SEQ ID NO: 118.

The disclosure further provides recombinant Clo051 endonucleases, or the nuclease domains thereof, comprising: an amino substitution at E90, F33, or a combination thereof relative to the amino acid sequence of SEQ ID NO: 71. In embodiments, the recombinant Clo051 endonuclease, or the nuclease domain thereof comprises up to 10 amino acid substitutions relative to the amino acid sequence of SEQ ID NO: 71.

The disclosure also provides recombinant Clo051 endonucleases, or the nuclease domains thereof, comprising: an amino substitution at E101, F44, or a combination thereof relative to the amino acid sequence of SEQ ID NO: 23. In embodiments, the recombinant Clo051 endonuclease, or the nuclease domain comprises up to 10 amino acid substitutions relative to the amino acid sequence of SEQ ID NO: 23.

The disclosure provides recombinant Clo051 endonucleases, or the nuclease domains thereof, comprising: an amino substitution at E478, F421, or a combination thereof relative to the amino acid sequence of SEQ ID NO: 117. In embodiments, the recombinant Clo051 endonuclease, or the nuclease domain thereof comprises up to 10 amino acid substitutions relative to the amino acid sequence of SEQ ID NO: 117.

The disclosure also provides recombinant Clo051 endonucleases, or the nuclease domains thereof, comprising: an amino substitution at E99, F42, or a combination thereof relative to the amino acid sequence of SEQ ID NO: 118. In embodiments, the recombinant Clo051 endonuclease, or the nuclease domain thereof comprises up to 10 amino acid substitutions relative to the amino acid sequence of SEQ ID NO: 118.

In embodiments, the Clo051 endonuclease, or a nuclease domain thereof comprises one or more amino acid substitutions. In embodiments, the Clo051 endonuclease, or a nuclease domain thereof comprises an amino substitution at E101. In embodiments, the Clo051 endonuclease, or a nuclease domain thereof comprises an amino substitution at F44. In embodiments, the Clo051 endonuclease, or a nuclease domain thereof comprises an amino substitution at E101 and an amino acid substitution at F44.

In embodiments, the amino substitution at E101 is E101S, E101N E101A, E101L, E101I, E101G, E101T, E101F, E101Y, E101W, E101P, E101H, E101Q, E101R, E101M, E101K, E101V, E101D, or E101C. In embodiments, the amino substitution at E101 is E101R, E101Q or E101K. In embodiments, the amino substitution at E101 is E101R. In embodiments, the amino substitution at F44 is F44S, F44T or F44A.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to any one of SEQ ID NOS: 72-90. In embodiments, the Clo051 endonuclease or a nuclease domain thereof comprises or consists of the amino acid sequence of any one of SEQ ID NOS: 72-90. In embodiments, the number of substitutions may be 1, 2, 3, 4, 5, 6, 7, 8, 9, 10 or up to 10.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 72. In embodiments, the Clo051 endonuclease or a nuclease domain thereof comprises or consists of the amino acid sequence of any one of SEQ ID NO: 72.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 73. In embodiments, the Clo051 endonuclease or a nuclease domain thereof comprises or consists of the amino acid sequence of any one of SEQ ID NO: 73.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 74. In embodiments, the Clo051 endonuclease or a nuclease domain thereof comprises or consists of the amino acid sequence of any one of SEQ ID NO: 74.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 75. In embodiments, the Clo051 endonuclease or a nuclease domain thereof comprises or consists of the amino acid sequence of any one of SEQ ID NO: 75.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 76. In embodiments, the Clo051 endonuclease or a nuclease domain thereof comprises or consists of the amino acid sequence of any one of SEQ ID NO: 76.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 77. In embodiments, the Clo051 endonuclease or a nuclease domain thereof comprises or consists of the amino acid sequence of any one of SEQ ID NO: 77.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 78. In embodiments, the Clo051 endonuclease or a nuclease domain thereof comprises or consists of the amino acid sequence of any one of SEQ ID NO: 78.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 79. In embodiments, the Clo051 endonuclease or a nuclease domain thereof comprises or consists of the amino acid sequence of any one of SEQ ID NO: 79.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 80. In embodiments, the Clo051 endonuclease or a nuclease domain thereof comprises or consists of the amino acid sequence of any one of SEQ ID No 80.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID No 81. In embodiments, the Clo051 endonuclease or a nuclease domain thereof comprises or consists of the amino acid sequence of any one of SEQ ID No 81.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 82. In embodiments, the Clo051 endonuclease or a nuclease domain thereof comprises or consists of the amino acid sequence of any one of SEQ ID NO: 82.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 83. In embodiments, the Clo051 endonuclease or a nuclease domain thereof comprises or consists of the amino acid sequence of any one of SEQ ID NO: 83.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 84. In embodiments, the Clo051 endonuclease or a nuclease domain thereof comprises or consists of the amino acid sequence of any one of SEQ ID NO: 84.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 85. In embodiments, the Clo051 endonuclease or a nuclease domain thereof comprises or consists of the amino acid sequence of any one of SEQ ID NO: 85.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 86. In embodiments, the Clo051 endonuclease or a nuclease domain thereof comprises or consists of the amino acid sequence of any one of SEQ ID NO: 86.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 87. In embodiments, the Clo051 endonuclease or a nuclease domain thereof comprises or consists of the amino acid sequence of any one of SEQ ID NO: 87.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 88. In embodiments, the Clo051 endonuclease or a nuclease domain thereof comprises or consists of the amino acid sequence of any one of SEQ ID NO: 88.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 89. In embodiments, the Clo051 endonuclease or a nuclease domain thereof comprises or consists of the amino acid sequence of any one of SEQ ID NO: 89.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 90. In embodiments, the Clo051 endonuclease or a nuclease domain thereof comprises or consists of the amino acid sequence of any one of SEQ ID NO: 90.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 117.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 23.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 71.

In embodiments, the Clo051 endonuclease or a nuclease domain thereof comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 118.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to any one of SEQ ID NOS: 92-110. In embodiments, the Clo051 endonuclease or a nuclease domain thereof is encoded by a nucleic acid sequence comprising or consisting of any one of SEQ ID NOS: 92-110.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 92. In embodiments, the Clo051 endonuclease or a nuclease domain thereof is encoded by a nucleic acid sequence comprising or consisting of SEQ ID NO: 92.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 93. In embodiments, the Clo051 endonuclease or a nuclease domain thereof is encoded by a nucleic acid sequence comprising or consisting of SEQ ID NO: 93.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 94. In embodiments, the Clo051 endonuclease or a nuclease domain thereof is encoded by a nucleic acid sequence comprising or consisting of SEQ ID NO: 94.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 95. In embodiments, the Clo051 endonuclease or a nuclease domain thereof is encoded by a nucleic acid sequence comprising or consisting of SEQ ID NO: 95.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 96. In embodiments, the Clo051 endonuclease or a nuclease domain thereof is encoded by a nucleic acid sequence comprising or consisting of SEQ ID NO: 96.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 97. In embodiments, the Clo051 endonuclease or a nuclease domain thereof is encoded by a nucleic acid sequence comprising or consisting of SEQ ID NO: 97.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 98. In embodiments, the Clo051 endonuclease or a nuclease domain thereof is encoded by a nucleic acid sequence comprising or consisting of SEQ ID NO: 98.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 99. In embodiments, the Clo051 endonuclease or a nuclease domain thereof is encoded by a nucleic acid sequence comprising or consisting of SEQ ID NO: 99.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 100. In embodiments, the Clo051 endonuclease or a nuclease domain thereof is encoded by a nucleic acid sequence comprising or consisting of SEQ ID NO: 100.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 101. In embodiments, the Clo051 endonuclease or a nuclease domain thereof is encoded by a nucleic acid sequence comprising or consisting of SEQ ID NO: 101.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 102. In embodiments, the Clo051 endonuclease or a nuclease domain thereof is encoded by a nucleic acid sequence comprising or consisting of SEQ ID NO: 102.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 103. In embodiments, the Clo051 endonuclease or a nuclease domain thereof is encoded by a nucleic acid sequence comprising or consisting of SEQ ID NO: 103.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 104. In embodiments, the Clo051 endonuclease or a nuclease domain thereof is encoded by a nucleic acid sequence comprising or consisting of SEQ ID NO: 104.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 105. In embodiments, the Clo051 endonuclease or a nuclease domain thereof is encoded by a nucleic acid sequence comprising or consisting of SEQ ID NO: 105.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 106. In embodiments, the Clo051 endonuclease or a nuclease domain thereof is encoded by a nucleic acid sequence comprising or consisting of SEQ ID NO: 106.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 107. In embodiments, the Clo051 endonuclease or a nuclease domain thereof is encoded by a nucleic acid sequence comprising or consisting of SEQ ID NO: 107.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 108. In embodiments, the Clo051 endonuclease or a nuclease domain thereof is encoded by a nucleic acid sequence comprising or consisting of SEQ ID NO: 108.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 109. In embodiments, the Clo051 endonuclease or a nuclease domain thereof is encoded by a nucleic acid sequence comprising or consisting of SEQ ID NO: 109.

In embodiments, the Clo051 endonuclease or a nuclease domain thereof is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 110. In embodiments, the Clo051 endonuclease or a nuclease domain thereof is encoded by a nucleic acid sequence comprising or consisting of SEQ ID NO: 110.

### Improved Cas-CLOVER Systems

The disclosure provides fusion proteins, comprising: (i) a DNA localization component, and (ii) any one of the Clo051 endonucleases disclosed herein or a nuclease domain thereof.

In embodiments, the DNA localization component comprises a DNA binding domain of a transcription activator-like effector (TALE). In embodiments, the DNA binding domain is derived from a Xanthomonas TALE or a Ralstonia TALE.

In embodiments, the DNA localization component comprises a catalytically inactive Cas protein, or a DNA binding domain thereof. Non-limiting examples of dCas proteins include dCas1, dCas1B, dCas2, dCas3, dCas4, dCas5, dCas6, dCas7, dCas8, dCas9, dCas10, dCas11 dCsy1, dCsy2, dCsy3, dCse1, dCse2, dCsc1, dCsc2, dCsa5, dCsn2, dCsm2, dCsm3, dCsm4, dCsm5, dCsm6, dCmr1, dCmr3, dCmr4, dCmr5, dCmr6, dCsb1, dCsb2, dCsb3, dCsx17, dCsx14, dCsx16, dCsaX, dCsx3, dCsx1, dCsx15, dCsf1, dCsf2, dCsf3, dCsf4, dCas12 (e.g., dCas12a, dCas12b, dCas12c, dCas12d, dCas12k, etc.), dCas13 (e.g., dCas13a, dCas13b (such as dCas13b-t1, dCas13b-t2, dCas13b-t3), dCas13c, dCas13d, etc.), dCas14, dCasX, dCasY, or any other variant of a naturally occurring Cas protein that is modified relative to its naturally-occurring counterpart effector protein to have reduced or eliminated catalytic activity relative to that of the naturally-occurring counterpart effector protein, but retains its ability to interact with a guide nucleic acid. Examples of dCas proteins that may be used with the systems disclosed herein include dCas proteins of Class 1 and Class 2 CRISPR-Cas systems.

In embodiments, the dCas protein is a dCas12, dCas12c2 or Cas12a. In embodiments, the dCas protein is a MAD7 protein, an engineered class 2 type V-A CRISPR-Cas (Cas12a/Cpf1) system isolated from Eubacterium rectale. In embodiments, the dCas9 is derived from Campylobacter jejuni Cas9 (CjCas9). In embodiments, the dCas9 is derived from Staphylococcus aureus (SaCas9). In embodiments, the dCas9 is derived from Streptococcus pyogenes (SpCas9). In embodiments, the dCas9 is derived from a Cas protein described in Casini A, et al., Nat Biotechnol. 2018 March;36(3):265-271; Slaymaker I M, et al. Science. 2016 Jan. 1;351(6268):84-8; Chen J S, et al. Nature 2017 Oct. 19;550(7676):407-410; Jinek M, et al. Science. 2012 Aug. 17;337(6096):816-21; Shams A, Nat Commun. 2021 Sep. 27;12(1):5664; and Kleinstiver B P, et al. Nature. 2016;529(7587):490-495. doi:10.1038/nature16526, the contents of each which is incorporated herein by reference in its entirety.

In embodiments, the catalytically inactive Cas protein is a catalytically inactive Cas9 (dCas9), or a catalytically inactive small Cas9 (dSaCas9).

In embodiments, the dCas9 comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 1. In embodiments, the dCas9 comprises or consists of the amino acid sequence of SEQ ID NO: 1.

In embodiments, the dSaCas9 comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 112. In embodiments, the dSaCas9 comprises or consists of the amino acid sequence of SEQ ID NO: 112.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to any one of SEQ ID NOS: 26-47. In embodiments, the fusion protein comprises or consists of the amino acid sequence of any one of SEQ ID NOS: 26-47.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 26. In embodiments, the fusion protein comprises or consists of the amino acid sequence of SEQ ID NO: 26.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 27. In embodiments, the fusion protein comprises or consists of the amino acid sequence of SEQ ID NO: 27.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 28. In embodiments, the fusion protein comprises or consists of the amino acid sequence of SEQ ID NO: 28.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 29. In embodiments, the fusion protein comprises or consists of the amino acid sequence of SEQ ID NO: 29.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 30. In embodiments, the fusion protein comprises or consists of the amino acid sequence of SEQ ID NO: 30.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 31. In embodiments, the fusion protein comprises or consists of the amino acid sequence of SEQ ID NO: 31.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 32. In embodiments, the fusion protein comprises or consists of the amino acid sequence of SEQ ID NO: 32.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 33. In embodiments, the fusion protein comprises or consists of the amino acid sequence of SEQ ID NO: 33.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 34. In embodiments, the fusion protein comprises or consists of the amino acid sequence of SEQ ID NO: 34.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 35. In embodiments, the fusion protein comprises or consists of the amino acid sequence of SEQ ID NO: 35.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 36. In embodiments, the fusion protein comprises or consists of the amino acid sequence of SEQ ID NO: 36.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 37. In embodiments, the fusion protein comprises or consists of the amino acid sequence of SEQ ID NO: 37.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 38. In embodiments, the fusion protein comprises or consists of the amino acid sequence of SEQ ID NO: 38.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 39. In embodiments, the fusion protein comprises or consists of the amino acid sequence of SEQ ID NO: 39.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 40. In embodiments, the fusion protein comprises or consists of the amino acid sequence of SEQ ID NO: 40.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 41. In embodiments, the fusion protein comprises or consists of the amino acid sequence of SEQ ID NO: 41.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 42. In embodiments, the fusion protein comprises or consists of the amino acid sequence of SEQ ID NO: 42.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 43. In embodiments, the fusion protein comprises or consists of the amino acid sequence of SEQ ID NO: 43.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 44. In embodiments, the fusion protein comprises or consists of the amino acid sequence of SEQ ID NO: 44.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 45. In embodiments, the fusion protein comprises or consists of the amino acid sequence of SEQ ID NO: 45.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 46. In embodiments, the fusion protein comprises or consists of the amino acid sequence of SEQ ID NO: 46.

In embodiments, the fusion protein comprises an amino acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 47. In embodiments, the fusion protein comprises or consists of the amino acid sequence of SEQ ID NO: 47.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to any one of SEQ ID NOS: 49-70. In embodiments, the fusion protein is encoded by a nucleic acid sequence comprising or consisting of any one of SEQ ID NOS: 49-70.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 49. In embodiments, the fusion protein is encoded by a nucleic acid sequence comprising or consisting of SEQ ID NO: 49.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 50. In embodiments, the fusion protein is encoded by a nucleic acid sequence comprising or consisting of SEQ ID NO: 50.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 51. In embodiments, the fusion protein is encoded by a nucleic acid sequence comprising or consisting of SEQ ID NO: 51.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 52. In embodiments, the fusion protein is encoded by a nucleic acid sequence comprising or consisting of SEQ ID NO: 52.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 53. In embodiments, the fusion protein is encoded by a nucleic acid sequence comprising or consisting of SEQ ID NO: 53.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 54. In embodiments, the fusion protein is encoded by a nucleic acid sequence comprising or consisting of SEQ ID NO: 54.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 55. In embodiments, the fusion protein is encoded by a nucleic acid sequence comprising or consisting of SEQ ID NO: 55.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 56. In embodiments, the fusion protein is encoded by a nucleic acid sequence comprising or consisting of SEQ ID NO: 56.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 57. In embodiments, the fusion protein is encoded by a nucleic acid sequence comprising or consisting of SEQ ID NO: 57.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 58. In embodiments, the fusion protein is encoded by a nucleic acid sequence comprising or consisting of SEQ ID NO: 58.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 59. In embodiments, the fusion protein is encoded by a nucleic acid sequence comprising or consisting of SEQ ID NO: 59.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 60. In embodiments, the fusion protein is encoded by a nucleic acid sequence comprising or consisting of SEQ ID NO: 60.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 61. In embodiments, the fusion protein is encoded by a nucleic acid sequence comprising or consisting of SEQ ID NO: 61.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 62. In embodiments, the fusion protein is encoded by a nucleic acid sequence comprising or consisting of SEQ ID NO: 62.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 63. In embodiments, the fusion protein is encoded by a nucleic acid sequence comprising or consisting of SEQ ID NO: 63.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 64. In embodiments, the fusion protein is encoded by a nucleic acid sequence comprising or consisting of SEQ ID NO: 64.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 65. In embodiments, the fusion protein is encoded by a nucleic acid sequence comprising or consisting of SEQ ID NO: 65.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 66. In embodiments, the fusion protein is encoded by a nucleic acid sequence comprising or consisting of SEQ ID NO: 66.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 67. In embodiments, the fusion protein is encoded by a nucleic acid sequence comprising or consisting of SEQ ID NO: 67.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 68. In embodiments, the fusion protein is encoded by a nucleic acid sequence comprising or consisting of SEQ ID NO: 68.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 69. In embodiments, the fusion protein is encoded by a nucleic acid sequence comprising or consisting of SEQ ID NO: 69.

In embodiments, the fusion protein is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 70. In embodiments, the fusion protein is encoded by a nucleic acid sequence comprising or consisting of SEQ ID NO: 70.

In embodiments, the fusion protein comprises a linker between the catalytically inactive Cas (e.g. dCas9), or the inactivated nuclease domain thereof, and the Clo051 endonuclease, or a nuclease domain thereof. The linker is not limited and may be any linker that can be used to bridge two proteins. For instance, the linker may be selected from Havlicek et al., Molecular Therapy, 2017, the contents of which are herein incorporated in its entirety for all purposes. In embodiments, the linker is a peptide linker. In embodiments, the peptide linker comprises the amino acid sequence of Gly-Gly-Gly-Gly-Ser (SEQ ID NO: 113). In embodiments, the fusion protein is capable of recognizing a protospacer adjacent motif (PAM) sequence on a target double stranded nucleic acid. In embodiments, the peptide linker comprises the amino acid sequence of any one or more of the following: SEQ ID Nos. 119-140.

In embodiments, the catalytically inactive Cas (e.g. dCas9) is capable of localizing to the nucleus. That is, in embodiments, the catalytically inactive Cas (e.g. dCas9) comprises a C-terminal SV40 nuclear localization sequence (NLS). In embodiments, the dCas9 comprising a C-terminal SV40 nuclear localization sequence (NLS) comprises an amino acid sequence having at least 70% (for example, about 75%, about 80%, about 85%, about 90%, about 95%, about 96%, about 97%, about 98%, about 99%, about 99.5%, or 100%, including all values and subranges that lie therebetween) identity to SEQ ID NO: 1. In embodiments, the dCas9 comprising a C-terminal SV40 nuclear localization sequence (NLS) comprises or consists of the amino acid sequence of SEQ ID NO: 1.

In embodiments, the dCas9 comprising a C-terminal SV40 nuclear localization sequence (NLS) is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 2. In embodiments, the dCas9 comprising a C-terminal SV40 nuclear localization sequence (NLS) is encoded by a nucleic acid sequence comprising or consisting of SEQ ID NO: 2.

In embodiments, the catalytically inactive Cas (e.g. dCas9) has limited ability to localize to the nucleus. In embodiments, the catalytically inactive Cas (e.g. dCas9) is not capable of localizing to the nucleus. For example, in embodiments, the catalytically inactive Cas (e.g. dCas9) lacks one or more amino acid residues of a C-terminal SV40 nuclear localization sequence (NLS). In embodiments, the catalytically inactive Cas (e.g. dCas9) lacks a C-terminal SV40 NLS. In embodiments, the dCas9 lacking a C-terminal SV40 nuclear localization sequence (NLS) comprises an amino acid sequence having at least 70% (for example, about 75%, about 80%, about 85%, about 90%, about 95%, about 96%, about 97%, about 98%, about 99%, about 99.5%, or 100%, including all values and subranges that lie therebetween) identity to SEQ ID NO: 114. In embodiments, the dCas9 lacking a C-terminal SV40 nuclear localization sequence (NLS) comprises or consists of the amino acid sequence of SEQ ID NO: 114.

In embodiments, the dCas9 lacking a C-terminal SV40 nuclear localization sequence is encoded by a nucleic acid sequence having at least about 70% identity (for example, about 75%, about 80%, about 85%, about 90%, about 95%, about 96%, about 97%, about 98%, about 99%, about 99.5%, or 100%, including all values and subranges that lie therebetween) to SEQ ID NO: 115. In embodiments, the dCas9 lacking a C-terminal SV40 nuclear localization sequence is encoded by a nucleic acid sequence comprising or consisting of SEQ ID NO: 115.

The disclosure further provides compositions, comprising: (a) a left guide RNA (gRNA) and a right gRNA; and (b) any one of the fusion proteins disclosed herein. In embodiments, the left gRNA is capable of binding to one strand of a target double stranded nucleic acid adjacent to a left protospacer adjacent motif (PAM) sequence, and the right gRNA is capable of binding to the other strand of the target double stranded nucleic acid adjacent to a right protospacer adjacent motif (PAM) sequence. In embodiments, the left gRNA and the fusion protein are capable of forming a left protein complex; and the right gRNA and the fusion protein are capable of forming a right protein complex. In embodiments, the Clo051 endonuclease or a nuclease domain thereof dimerizes resulting in a heterodimer of the left protein complex and the right protein complex.

In embodiments, the fusion protein is capable of recognizing the left PAM sequence and the right PAM sequence on the target double stranded nucleic acid. In embodiments, the composition is capable of catalyzing a double stranded break in the target nucleic acid. In embodiments, the double stranded break is located between the left PAM sequence and the right PAM sequence on the target double stranded nucleic acid.

Without being bound to a theory, it is thought that few, if any, double stranded breaks are catalyzed by either the left protein complex or the right protein complex alone. Rather, it is thought that dimerization of the left and the right complexes promotes the catalysis of double stranded breaks in the target nucleic acid, which advantageously enhances the stringency of the disclosed Cas-CLOVER gene editing systems and reduces off-target activity, while improving cutting efficiency.

In embodiments, the 5′ end of the left gRNA and/or the 5′ end of the right gRNA are conjugated to a tRNA linker. In embodiments, the tRNA linker comprises a nucleic acid sequence of SEQ ID NO: 111. Methods of conjugating tRNA linkers to gRNAs and further details and examples of tRNA linkers are provided in Xie K, et al. Proc Natl Acad Sci USA. 2015 Mar. 17;112(11):3570-5, which is incorporated herein by reference in its entirety for all purposes. In embodiments, a polycistronic tRNA-gRNA (PTG) gene is designed, which is transcribed into a primary transcript comprising tandem repeats of tRNA-gRNA. This primary transcript is processed by endogenous tRNA-processing RNases (e.g., RNase P and RNase Z in plants) to excise the individual gRNAs from the PTG transcript. The resulting gRNAs (e.g. left and right gRNAs) are capable of directing the Cas-CLOVER systems disclosed herein to the target nucleic acid.

### Methods of Using the Improved Cas-CLOVER Systems

The disclosure provides methods of introducing a double stranded break in a target nucleic acid, the method comprising: bringing any one of the compositions disclosed herein in contact with the target nucleic acid.

In embodiments, the cutting efficiency of the composition is higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease, or a nuclease domain thereof. In embodiments, the cutting efficiency is measured using the ADE2 reporter assay.

In embodiments, the cutting efficiency of the composition is higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease, or a nuclease domain thereof.

In embodiments, the C-terminal SV40 nuclear localization (NLS) sequence of the dCas9 is deleted in the control fusion protein.

In embodiments, the cutting efficiency of the composition is higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the cutting efficiency of the composition is higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the cutting efficiency of the composition is more than about 50% (for example, about 55%, about 60%, about 65%, about 70%, about 75%, about 80%, about 85%, about 90%, about 95% about 96%, about 97%, about 98%, about 99%, or 100%, including all values and subranges that lie therebetween). In embodiments, the cutting efficiency of the composition is more than about 80%.

In embodiments, the cutting efficiency of the composition is at least about 1.5 fold (for example, about 2 fold, about 3 fold, about 4 fold, about 5 fold, about 10 fold, about 20 fold, about 50 fold, about 70 fold, about 100 fold, about 200 fold, about 300 fold, about 400 fold, about 500 fold, about 6000 fold, about 700 fold, about 800 fold, about 900 fold, about 1000 fold, or about 10,000 fold) higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.

In embodiments, the cutting efficiency of the composition is at least about 1.5 fold (for example, about 2 fold, about 3 fold, about 4 fold, about 5 fold, about 10 fold, about 20 fold, about 50 fold, about 70 fold, about 100 fold, about 200 fold, about 300 fold, about 400 fold, about 500 fold, about 6000 fold, about 700 fold, about 800 fold, about 900 fold, about 1000 fold, or about 10,000 fold) higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof.

In embodiments, the cutting efficiency of the composition is at least about 1.5 fold (for example, about 2 fold, about 3 fold, about 4 fold, about 5 fold, about 10 fold, about 20 fold, about 50 fold, about 70 fold, about 100 fold, about 200 fold, about 300 fold, about 400 fold, about 500 fold, about 6000 fold, about 700 fold, about 800 fold, about 900 fold, about 1000 fold, or about 10,000 fold) higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the cutting efficiency of the composition is at least about 1.5 fold (for example, about 2 fold, about 3 fold, about 4 fold, about 5 fold, about 10 fold, about 20 fold, about 50 fold, about 70 fold, about 100 fold, about 200 fold, about 300 fold, about 400 fold, about 500 fold, about 6000 fold, about 700 fold, about 800 fold, about 900 fold, about 1000 fold, or about 10,000 fold) higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the cutting efficiency of the composition is at least about 5% (for example, about 10%, about 20%, about 30%, about 40%, about 50%, about 60%, about 70%, about 80%, about 90%, about 100%, about 200%, about 500%, about 700% or about 1000%) higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.

In embodiments, the cutting efficiency of the composition is at least about 5% (for example, about 10%, about 20%, about 30%, about 40%, about 50%, about 60%, about 70%, about 80%, about 90%, about 100%, about 200%, about 500%, about 700% or about 1000%) higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof.

In embodiments, the cutting efficiency of the composition is at least about 5% (for example, about 10%, about 20%, about 30%, about 40%, about 50%, about 60%, about 70%, about 80%, about 90%, about 100%, about 200%, about 500%, about 700% or about 1000%) higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the cutting efficiency of the composition is at least about 5% (for example, about 10%, about 20%, about 30%, about 40%, about 50%, about 60%, about 70%, about 80%, about 90%, about 100%, about 200%, about 500%, about 700% or about 1000%) higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

In embodiments, the contacting occurs in vitro, in vivo, or ex vivo. In embodiments, the contacting occurs within a cell. The type of cell is not limited, and may be a microbial cell, a fungal cell, a plant cell, or an animal cell. In embodiments, the animal cell is a mammalian cell. In embodiments, the microbial cell is a bacterial cell. In embodiments, the fungal cell is a yeast cell. In embodiments, the plant cell is a banana plant cell or a tobacco plant cell.

In embodiments, the cellular toxicity of the composition is lower than, or the same as, a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof. In embodiments, the cellular toxicity is measured using the ADE2 reporter assay. In embodiments, the cellular toxicity of the composition is at least 5% (for example, about 10%, about 20% about 30% about 40%, about 50%, about 60%, about 70%, about 80%, about 90%, or about 100%) less than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.

In embodiments, the cellular toxicity of the composition is lower than, or the same as, a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71. In embodiments, the cellular toxicity of the composition is at least 5% (for example, about 10%, about 20% about 30% about 40%, about 50%, about 60%, about 70%, about 80%, about 90%, or about 100%) less than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a Clo051 endonuclease, or a nuclease domain thereof comprising the amino acid sequence of SEQ ID NO. 23, 118, 117 or 71.

The disclosure further provides methods of modifying a target double stranded nucleic acid, comprising: bringing (a) any one of the compositions disclosed herein and (b) a donor nucleic acid, in contact with the target nucleic acid, wherein the donor nucleic acid is capable of homologous recombination with the target nucleic acid. In embodiments, the contacting occurs in vitro, in vivo, or ex vivo. In embodiments, the contacting occurs in vivo, and the composition and the donor nucleic acid are administered to a subject, in need thereof. In embodiments, the subject is a human subject.

In embodiments, the donor nucleic acid is integrated into the target nucleic acid through homologous recombination or non-homologous end-joining (NHEJ). In embodiments, the methods of modifying a target double stranded nucleic acid disclosed herein comprise replacing, inserting or deleting a gene, or a fragment thereof; or a regulatory sequence or a fragment thereof. In embodiments, the methods of modifying a target double stranded nucleic acid disclosed herein comprise correcting or creating one or more loss or gain of function mutations, deletions, or translocations associated with disease states or disorders or traits or phenotypes. Thus, the methods of modifying a target double stranded nucleic acid disclosed herein may be used to create desired phenotypes or traits, biomanufacturing, biosynthesis or treat disease states or disorders in subjects.

In embodiments, the donor nucleic acid is used to edit the target nucleic acid. In embodiments, the integration of the donor nucleic acid introduces one or more nucleotide mutations into the target nucleic acid. In embodiments, the donor nucleic acid comprises one or more mutations to be introduced into the target nucleic acid. The one or more mutations introduced by the donor nucleic acid may be one or more substitutions, deletions, insertions, or a combination thereof. The mutations may cause a shift in an open reading frame on the target nucleic acid. In embodiments, the donor nucleic acid delivers a transgene to the target nucleic acid. In embodiments, the donor nucleic acid alters a stop codon in the target nucleic acid. For example, the donor nucleic acid may correct a premature stop codon. The correction may be achieved by deleting the stop codon or introducing one or more mutations to the stop codon. In embodiments, the integration of the donor nucleic acid disrupts, restores or introduces a splicing site.

### EXAMPLES

The following examples, which are included herein for illustration purposes only, are not intended to be limiting.

### Example 1: Methods of Identifying Mutant dCas9-Clo051 Fusion Proteins with Enhanced Cutting Efficiency and Lowered Cellular Toxicity

Although the Cas-CLOVER gene editing system, which uses the dCas9-Clo051 fusion protein, can catalyze the formation of double stranded breaks in DNA resulting in homologous recombination of a donor nucleic acid at a target site (FIG. 1A), the cutting efficiency is typically low (less than 50%). Moreover, the expression of dCas9-Clo051, particularly from strong promoters, can be toxic to cells. To generate improved Cas-CLOVER gene editing systems that have increased cutting efficiency and lower cellular toxicity, mutant variants of the dCas9-Clo051 fusion protein, comprising mutant versions of the Clo051 nuclease domain, were generated, and their gene editing capability and cellular toxicity were tested using the ADE2 reporter assay, as described below.

In the ADE2 reporter assay, an yeast strain of the genotype MATa, ura3Δ0, leu2Δ0 is grown and made competent using the following method. Competent yeast cells are made by culturing cells and treating them in accordance with the Zymo Research Frozen-EZ Transformation II Kit (Cat#T2001). The competent cells were then transformed with a plasmid under leucine selection encoding a particular mutant version dCas9-Clo051 protein or unmodified (control) dCas9-Clo051 protein, along with a left gRNA, a right gRNA and a donor nucleic acid that was designed to be homologous to the regions that flank the ADE2 coding sequence in the yeast genome (FIG. 1B). The sample was mixed with 250 μl of EZ Transformation Solution III, and then incubated at 30° C. for 60 minutes. Thereafter, the sample was plated on selection media lacking leucine and having half the amount of adenine and incubated at 30° C. for 4 days.

Successful homologous recombination of the donor nucleic acid at the target site through the activity of the Cas-CLOVER system results in the deletion of the ADE2 coding sequence, and thereby, causes accumulation of the adenine precursor (aminoimidazoleribotide, or AIR) in the vacuoles. AIR is aerobically oxidized by the cells to a red pigment, thereby leading to red coloration of the yeast colonies. The percentage of red colonies among the transformants on the plate gives the “% cutting efficiency” of the Cas-CLOVER system that was used in that transformation, such that, a higher number of red colonies indicates enhanced cutting efficiency of the tested Cas-CLOVER system. Additionally, the total number of transformants obtained was noted, which is inversely correlated to the cellular toxicity of the Cas-CLOVER system that was used in that transformation. That is, the transformation of a less toxic Cas-CLOVER system is expected to give rise to higher number of yeast colonies, and vice versa. If needed, further analysis of colonies from plates was done using ImageJ and AzureSpot Pro and GraphPad.

### Example 2: Substitution of Amino Acid F44 of Clo051, or the Nuclease Domain Thereof, Improves the Cutting Efficiency and Lowers the Cellular Toxicity of the Cas-CLOVER System

To test whether substitutions at the amino acid F44 of Clo051, or a nuclease domain thereof, would improve the gene editing capabilities of the Cas-CLOVER system, mutant versions of the nuclease domain of Clo051, comprising either one of the amino acid substitutions: F44S, F44T or F44A, were generated and fused to the dCas9 protein to generate mutant dCas9-Clo051 fusion proteins (SEQ ID NOS: 5, 6 and 7).

Using the methods described in Example 1, each of these mutant dCas9-Clo051 fusion proteins or the control dCas9-Clo051 protein (comprising the nuclease domain of Clo051 having amino acid sequence of SEQ ID NO: 23) was expressed in yeast cells under the control of the high strength promoter, ADH1, together with a left gRNA and a right gRNA. A donor nucleic acid designed to be homologous to the regions that flank the ADE2 coding sequence in the yeast genome was also introduced into the cells. The ADE2 reporter assay was performed and the transformants were analyzed.

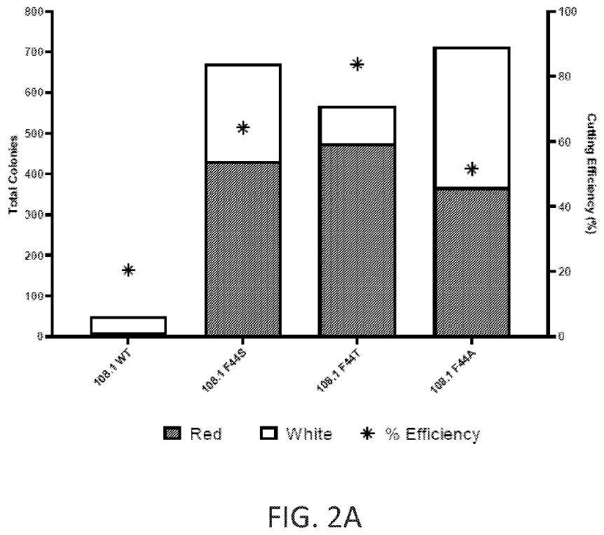

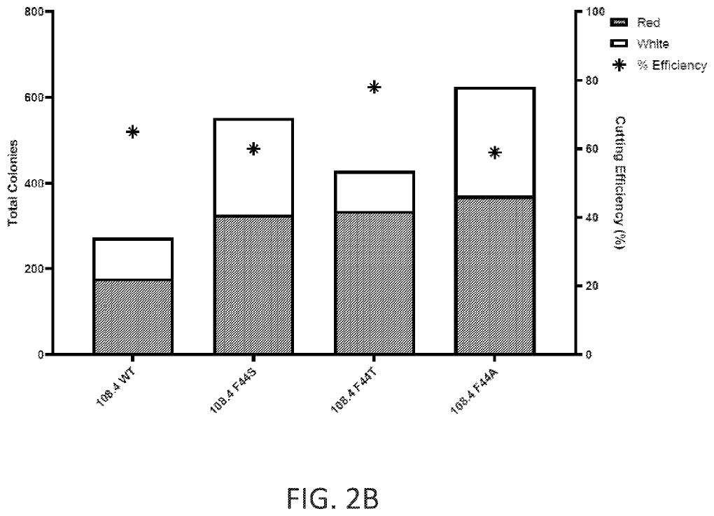

As shown in FIG. 2A, a higher number of colonies was obtained with the expression of F44S, F44T or F44A mutant dCas9-Clo051 fusion proteins, as compared to the control dCas9-Clo051 fusion protein (“108.1 WT”), indicating that the F44S, F44T or F44A mutant dCas9-Clo051 fusion proteins have lower cellular toxicity compared to the control dCas9-Clo051 fusion protein, even upon expression of the mutant dCas9-Clo051 fusion proteins using the high strength ADH1 promoter. Furthermore, the cutting efficiency of the F44S, F44T or F44A mutant dCas9-Clo051 fusion protein was improved by about 44%, 63% and 31%, respectively, as compared to the control dCas9-Clo051 fusion protein. Furthermore, as shown in FIG. 2B, expression of these dCas9-Clo051 fusion proteins in yeast cells under the control of a low strength REV1 promoter also resulted in similar reduction in cellular toxicity, as compared to REV1-mediated expression of the control dCas9-Clo051 fusion protein (“108.4 WT”).

Thus, the results described above demonstrate that substitution of the amino acid F44 in Clo051, particularly with S, T or A, enhances the cutting efficiency while lowering the cellular toxicity of the Cas-CLOVER system.

It is surprising and unexpected that the amino acid substitutions in the nuclease domain of Clo051 disclosed herein improve Cas-CLOVER gene editing capabilities because mutating Clo051 based on amino acid substitutions that enhance the DNA cleavage capabilities of another Type II endonuclease, FokI, did not meet with much success. In other words, while certain amino acid substitutions in the Type IIS endonuclease, FokI suppressed off-target cleavage and improved on-target cleavage frequency of zinc finger nucleases comprising the mutant FokI, the equivalent amino acid substitutions in Clo051 did not lead to improved cutting efficiency of Clo051. Further details on amino acid mutations that improved FokI function are provided in Miller JC, et al. Nat Biotechnol. 2019 August;37(8):945-952; Miller J C, et al. Nat Biotechnol. 2007 July;25(7):778-85, and Doyon Y, et al. Nat Methods. 2011 January;8(1):74-9, the contents of each of which is incorporated by reference in its entirety for all purposes.

For example, the amino acid substitutions Q481A and I479Q in FokI suppress off-target cleavage while enhancing on-target cleavage frequency. However, mutant versions of Clo051 comprising the amino acid substitutions Q109A or 1107Q (which are equivalent to Q481A and I479Q in FokI) did not show enhanced cutting when used in a Cas-CLOVER system; in fact, cutting efficiency was adversely affected with the use of these Clo051 mutants. Similarly other Clo051 mutants-R50H, S104G, S104D and K153S (which are equivalent to R422H, N476G, N476D, and K525S in FokI)—also adversely affected Clo051 cutting efficiency in the Cas-CLOVER system.

In sum, the comparative data with FokI described above further underscore the superior and surprising effects of the amino acid substitutions of the nuclease domain of Clo051 disclosed herein that significantly improve the gene editing functions of the Cas-CLOVER system.

### Example 3: Substitution of Amino Acid E101 of Clo051, or the Nuclease Domain Thereof, Improves the Cutting Efficiency of the Cas-CLOVER System

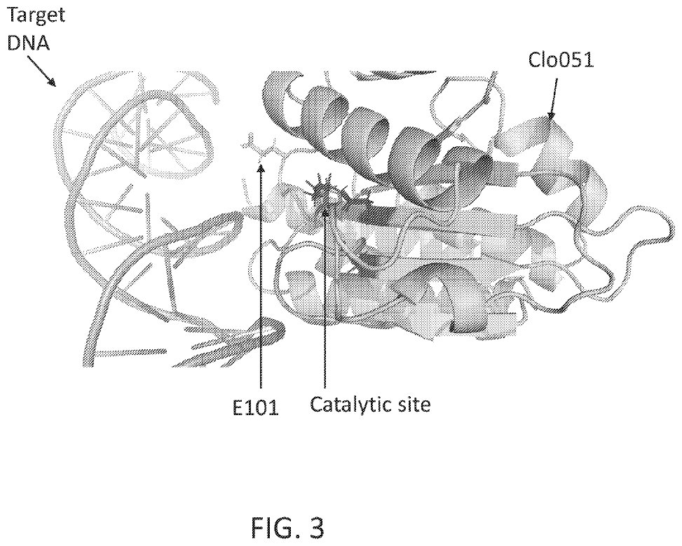

Amino acids E101, Y99 and Y103, among other amino acids, are located near the catalytic site and the target nucleic acid-binding site of Clo051 (FIG. 3). A structural model of Clo051 generated using PHYRE2 (described in Kelley L A et al. Nature Protocols 10, 845-858 (2015), which is incorporated herein by reference in its entirety for all purposes) and the FokI structure from the Protein Data Bank were aligned using PyMOL. To test whether substitutions at the amino acid E101 of Clo051 would improve the gene editing capabilities of the Cas-CLOVER system, mutant versions of the nuclease domain of Clo051, comprising either one of the amino acid substitutions: E101S, E101N, or E101A, were generated and fused to the dCas9 protein to generate mutant dCas9-Clo051 fusion proteins.

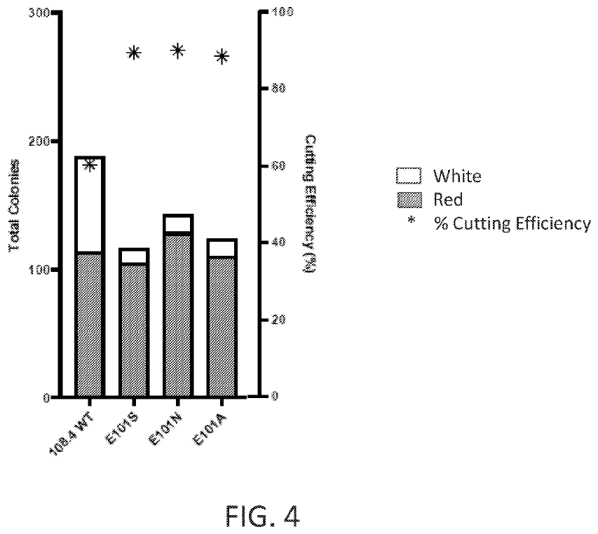

Using the methods described in Example 1, these mutant dCas9-Clo051 fusion proteins were expressed in yeast cells under the control of the low strength promoter, ScREV1, together with a left gRNA, and a right gRNA. A donor nucleic acid designed to be homologous to the regions that flank the ADE2 coding sequence in the yeast genome was also introduced into the cells. The ADE2 reporter assay was performed and the transformants were analyzed. As shown in FIG. 4, the cutting efficiency was markedly increased (by about 20-30%) upon expression of E101S, E101N, or E101A mutant dCas9-Clo051 fusion proteins, as compared to the control dCas9-Clo051 fusion protein (comprising the nuclease domain of Clo051 having amino acid sequence of SEQ ID NO: 23).

Thus, the results described above demonstrate that the substitution of amino acid E101 in the nuclease domain of Clo051, for example, with S, N or A, enhances the cutting efficiency of the Cas-CLOVER system. Notably, while substituting amino acid E101 the nuclease domain of Clo051 resulted in enhanced cutting efficiency, substitution of amino acid Y99 or Y103 abolished gene editing by the Cas-CLOVER system. This underscores the superior and unexpected effect of mutating the E101 residue, as disclosed herein.

### Example 4: Combining Modifications of the Nuclease Domain of Clo051, dCas9 and the Guide RNA Markedly Improves the Cutting Efficiency and Lowers Cellular Toxicity of the Cas-CLOVER System

In the following experiments, the 5′ end of the left gRNA and the 5′ end of the right gRNA was each conjugated to a tRNA linker, comprising a nucleic acid sequence of SEQ ID NO: 111, and dCas9 was modified by deleting its C-terminal SV40 nuclear localization (NLS) sequence. Mutant dCas9-Clo051 fusion proteins, comprising: (i) F44 or E101 substitutions in the nuclease domain of Clo051 and (ii) deletion of the C-terminal SV40 NLS of dCas9 were expressed in yeast cells along with the 5′ tRNA linker-conjugated left gRNA and 5′ tRNA linker-conjugated right gRNA. A donor nucleic acid designed to be homologous to the regions that flank the ADE2 coding sequence in the yeast genome was also introduced into the cells.

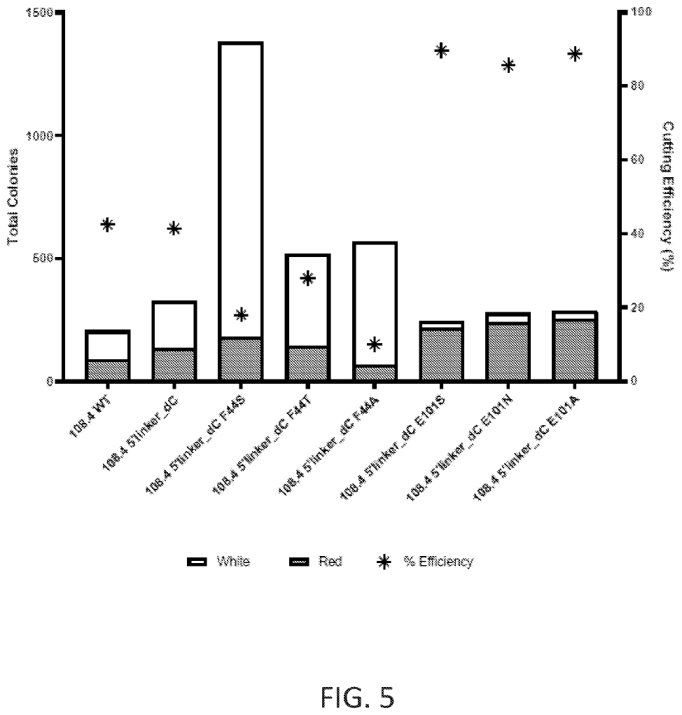

Remarkably, as shown in FIG. 5, the cutting efficiency of Cas-CLOVER systems comprising: (i) a mutant nuclease domain of Clo051 comprising an E101 amino acid substitution of E101S, E101N or E101A, (ii) C-terminal SV40 NLS-deleted dCas9 and (iii) 5′ tRNA linker-conjugated guide RNAs was more than about 80% and was accompanied by a reduction in cellular toxicity, as compared to the control Cas-CLOVER system, having an unmodified dCas9-Clo051 fusion protein (comprising the nuclease domain of Clo051 having amino acid sequence of SEQ ID NO: 23) and unmodified gRNAs.

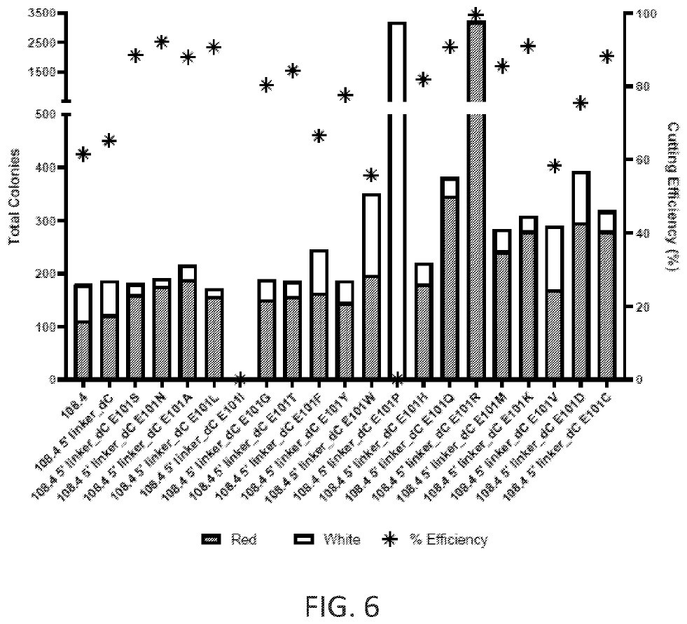

Next, mutant dCas9-Clo051 fusion proteins, comprising: (i) substitution of E101 in the nuclease domain of Clo051 with any other amino acid and (ii) deletion of the C-terminal SV40 NLS of dCas9 was evaluated in combination with (iii) 5′ tRNA linker-conjugated guide RNA. Notably, 12 out of the 19 tested E101 mutants gave rise to extremely high cutting efficiencies (over 80%), without significant effects on cellular toxicity (FIG. 6). Most unexpectedly, the amino acid substitution of E101R in the nuclease domain of Clo051 in combination with the deletion of the C-terminal SV40 NLS from dCas9 and the 5′ tRNA linker-conjugated guide RNAs resulted in near 100% cutting efficiency and markedly reduced cellular toxicity.

These results demonstrate that amino acid modification of E101 in the nuclease domain of Clo051 in combination with the deletion of the C-terminal SV40 NLS of dCas9 and the use of 5′ tRNA linker-conjugated guide RNAs gives rise to a significantly improved Cas-CLOVER system with enhanced gene editing capability and reduced cellular toxicity, compared to the control Cas-CLOVER system.

### Example 5: Combining Modifications of the Nuclease Domain of Clo051, dCas9 and the Guide RNA Lowers Cellular Toxicity

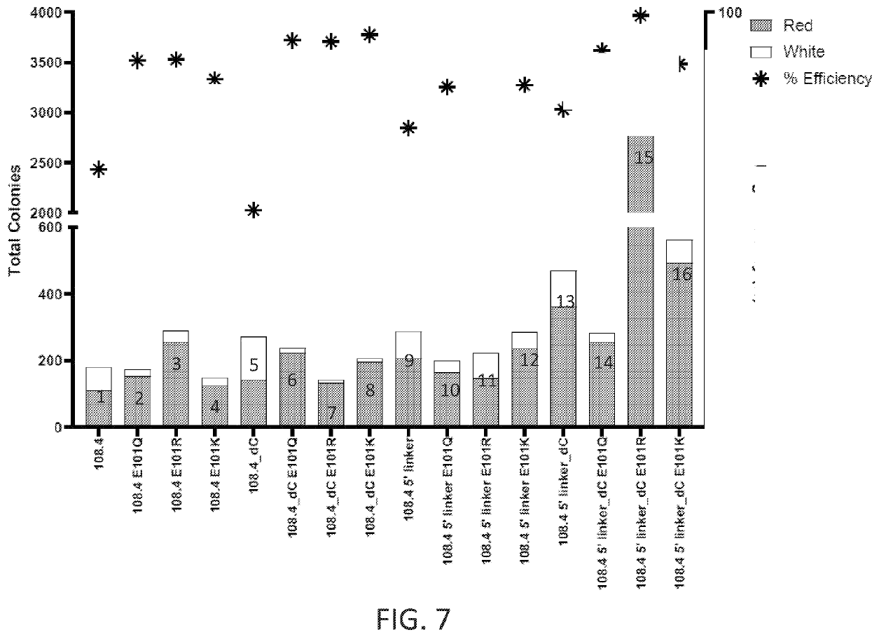

In FIG. 7, the cellular toxicity of the Cas CLOVER system was evaluated as described in Example 1 for each of the systems listed on the X axis. Notably, Cas-CLOVER systems comprising a mutant nuclease domain of Clo051 comprising an E101 amino acid substitution of E101Q (sample 2), E101R, (sample 3) or E101K (sample 4) exhibited comparable cellular toxicity relative to Cas-CLOVER systems comprising a nuclease domain of Clo051 having amino acid sequence of SEQ ID NO: 23 (“108.4”; sample 1).

Additionally, the following Cas CLOVER systems showed minimal change in cellular toxicity, as compared to Cas-CLOVER systems comprising a wild type Clo051 (“108.4”; sample 1):

Sample 5: Cas CLOVER systems comprising a nuclease domain of Clo051 having the amino acid sequence of SEQ ID NO: 23 and a C-terminal SV40 NLS-deleted dCas9;

Samples 6-8: Cas CLOVER systems comprising a mutant Clo051 nuclease domain comprising an E101 amino acid substitution of E101Q, E101R or E101K and a C-terminal SV40 NLS-deleted dCas9;

Sample 9: Cas CLOVER systems comprising 5′ tRNA linker-conjugated guide RNAs, and a nuclease domain of Clo051 having the amino acid sequence of SEQ ID NO: 23;

Samples 10-12: Cas CLOVER systems comprising 5′ tRNA linker-conjugated guide RNAs in combination with a mutant Clo051 nuclease domain comprising an E101 amino acid substitution of E101Q, E101R or E101K; and

Sample 13: Cas CLOVER systems comprising a nuclease domain of Clo051 having amino acid sequence of SEQ ID NO: 23 and a C-terminal SV40 NLS-deleted dCas9.

FIG. 7 shows that in sample 15, the combination of: (i) E101R substitution in Clo051 nuclease domain, (ii) deletion of the C-terminal SV40 NLS of dCas9, and (iii) 5′ tRNA linker-conjugated guide RNA has a synergistically improved effect on cellular toxicity, with remarkably higher number of colonies. Furthermore, in sample 16, a combination of: (i) E101K substitution in Clo051 nuclease domain, (ii) deletion of the C-terminal SV40 NLS of dCas9, and (iii) 5′ tRNA linker-conjugated guide RNA also gave rise to a higher number of colonies, as compared to the control Cas CLOVER systems without the modifications.

### Example 6: Marked Enhancement of Cutting Efficiency of Cas-CLOVER Systems Having E101 Substitutions in the Clo051 Nuclease Domain

To evaluate whether the substitution of E101, for instance, with Q, R or K, is sufficient to markedly improve cutting efficiency of the Cas CLOVER system, the following experiment was performed. The results showed that substitution of E101 of the Clo051 nuclease domain with either Q, R or K results in remarkably higher cutting efficiency of the Cas CLOVER systems.

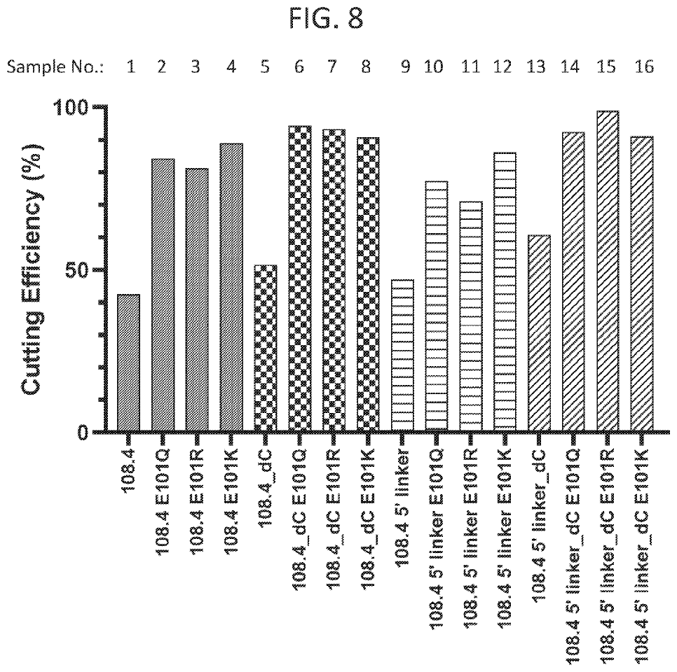

As shown in FIG. 8, Cas CLOVER systems comprising a mutant Clo051 nuclease domain comprising an E101 amino acid substitution of E101Q, E101R or E101K (samples 2-4) exhibited remarkably increased cutting efficiency, as compared to Cas-CLOVER systems comprising a nuclease domain of Clo051 having the amino acid sequence of SEQ ID NO: 23 (“108.4”; sample 1).

Furthermore, the results show that the improved Clo051 mutants described here are compatible with other Cas CLOVER modifications, such as, deletion of C-terminal SV40 NLS in dCas9 and the use of 5′ tRNA linker-conjugated guide RNAs, as demonstrated by the cutting efficiency of the following Cas-CLOVER systems depicted in FIG. 8: (i) Cas CLOVER systems comprising a mutant Clo051 nuclease domain comprising an E101 amino acid substitution of E101Q, E101R or E101K in combination with a C-terminal SV40 NLS-deleted dCas9 (samples 6-8); (ii) Cas CLOVER systems comprising 5′ tRNA linker-conjugated guide RNAs in combination with a mutant Clo051 nuclease domain comprising a substitution of E101 with either Q, R or K (samples 10-12); or (iii) Cas CLOVER systems comprising 5′ tRNA linker-conjugated guide RNAs in combination with a mutant Clo051 nuclease domain comprising an E101 amino acid substitution of E101Q, E101R or E101K and a dCas9 comprising a C-terminal SV40 NLS-deleted dCas9 (samples 14-16). While sample 5 is a control Cas CLOVER system comprising a nuclease domain of Clo051 having amino acid sequence of SEQ ID NO: 23 in combination with a dCas9 comprising a C-terminal SV40 NLS, sample 9 is a control Cas CLOVER system comprising 5′ tRNA linker-conjugated guide RNAs and a nuclease domain of Clo051 having amino acid sequence of SEQ ID NO: 23, and sample 13 is a control Cas CLOVER system comprising 5′ tRNA linker-conjugated guide RNAs, a nuclease domain of Clo051 having amino acid sequence of SEQ ID NO: 23 and a dCas9 comprising a C-terminal SV40 NLS.

These data clearly demonstrate that the substitution of E101, for instance, with Q, R or K, results in remarkably higher cutting efficiency of the Cas CLOVER systems, indicating that the modification of this residue of the Clo051 nuclease domain produces a highly effective tool for genetic engineering.

### Example 7: Gene Editing of Mammalian and Plant Systems Using The Improved Cas-CLOVER System

Using the methods of gene editing disclosed herein, the improved Cas-CLOVER systems disclosed herein are used to generate double stranded breaks and optionally, edit the genome of mammalian cells, such as, Chinese hamster ovary (CHO) cells; and plant cells, such as, tobacco cells and banana cells. The gene editing is done in vitro, ex vivo and in vivo. For instance, the improved Cas-CLOVER systems disclosed herein are used to generate double stranded breaks and optionally, edit the genome of plants, such as, tobacco and banana; animals, such as, mice and rats; and humans.

Gene editing (e.g. knockout mutation of the phytoene desaturase (PDS) gene) using the improved Cas-CLOVER systems disclosed herein in plants, such as, banana and in mammalian systems will be done using methods described in Tripathi, L., Ntui, V., Tripathi, J., Norman, D., Crawford, J. (2023) A new and novel high-fidelity genome editing tool for banana using Cas-CLOVER. Plant Biotech. J.; Madison, B., Patil, D., Richter, M., Li, X., Cranert, S., Wang, X., Martin, R., Xi, H., Tan, Y., Weiss, L, Marquez, K., Coronella, J., Shedlock, Ostertag, E. (2022) Cas-CLOVER is a novel high-fidelity nuclease for safe and robust generation of TSCM-enriched allogeneic CAR-T cells. Mol. Thera. Nuc. Acids.; Chen L, et al. FEBS Open Bio. 2021 July;11(7):1965-1980; Liu W H, et al. Sci Rep. 2021 Jun. 16;11(1):12649; and Jung S B, et al. Nucleic Acids Res. 2021 Sep. 7;49(15):e85.

The foregoing is illustrative of the present invention, and is not to be construed as limiting thereof. The invention is defined by the following claims, with equivalents of the claims to be included therein.

### Numbered Embodiments

The following list of embodiments is included herein for illustration purposes only and is not intended to be comprehensive or limiting. The subject matter to be claimed is expressly not limited to the following embodiments.
Embodiment 1. A recombinant Clo051 endonuclease, or a nuclease domain thereof, comprising:
   (i) an amino substitution at E101 of SEQ ID NO: 23, or at a corresponding amino acid residue, of a wild type Clo051 endonuclease or a nuclease domain thereof,
   (ii) an amino substitution at F44 of SEQ ID NO: 23, or at a corresponding amino acid residue, of a wild type Clo051 endonuclease or a nuclease domain thereof, or
   (iii) a combination thereof.
Embodiment 2. A recombinant Clo051 endonuclease, or a nuclease domain thereof, comprising:
   (i) an amino substitution at E101 of SEQ ID NO: 23, or at the corresponding amino acid residue of SEQ ID NO: 71, SEQ ID NO: 117 or SEQ ID NO: 118,
   (ii) an amino substitution at F44 of SEQ ID NO: 23, or at the corresponding amino acid residue of SEQ ID NO: 71, SEQ ID NO: 117 or SEQ ID NO: 118, or
   (iii) a combination thereof.
Embodiment 3. The recombinant Clo051 endonuclease, or the nuclease domain thereof of embodiment 1, wherein the recombinant Clo051 endonuclease, or the nuclease domain thereof comprises up to 10 amino acid substitutions relative to SEQ ID NO: 23 or the amino acid sequence of a wild type Clo051 endonuclease or a nuclease domain thereof.
Embodiment 4. The recombinant Clo051 endonuclease, or the nuclease domain thereof of embodiment 2, wherein the recombinant Clo051 endonuclease, or the nuclease domain thereof comprises up to 10 amino acid substitutions relative to SEQ ID NO: 23, SEQ ID NO: 71, SEQ ID NO: 117 or SEQ ID NO: 118.
Embodiment 5. The recombinant Clo051 endonuclease, or the nuclease domain thereof of any one of embodiments 1-4, comprising: an amino substitution at E90, F33, or a combination thereof relative to the amino acid sequence of SEQ ID NO: 71.
Embodiment 6. The recombinant Clo051 endonuclease, or the nuclease domain thereof of any one of embodiments 1-5, comprising: an amino substitution at E101, F44, or a combination thereof relative to the amino acid sequence of SEQ ID NO: 23.
Embodiment 7. The recombinant Clo051 endonuclease, or the nuclease domain thereof of any one of embodiments 1-6, comprising: an amino substitution at E478, F421, or a combination thereof relative to the amino acid sequence of SEQ ID NO: 117.
Embodiment 8. The recombinant Clo051 endonuclease, or the nuclease domain thereof of any one of embodiments 1-7, comprising: an amino substitution at E99, F42, or a combination thereof relative to the amino acid sequence of SEQ ID NO: 118.
Embodiment 9. A recombinant Clo051 endonuclease, or the nuclease domain thereof, comprising: an amino substitution at E90, F33, or a combination thereof relative to the amino acid sequence of SEQ ID NO: 71.
Embodiment 10. The recombinant Clo051 endonuclease, or the nuclease domain thereof of embodiment 9, comprising up to 10 amino acid substitutions relative to the amino acid sequence of SEQ ID NO: 71.
Embodiment 11. A recombinant Clo051 endonuclease, or the nuclease domain thereof, comprising: an amino substitution at E101, F44, or a combination thereof relative to the amino acid sequence of SEQ ID NO: 23.
Embodiment 12. The recombinant Clo051 endonuclease, or the nuclease domain thereof of embodiment 11, comprising up to 10 amino acid substitutions relative to the amino acid sequence of SEQ ID NO: 23.
Embodiment 13. A recombinant Clo051 endonuclease, or the nuclease domain thereof, comprising: an amino substitution at E478, F421, or a combination thereof relative to the amino acid sequence of SEQ ID NO: 117.
Embodiment 14. The recombinant Clo051 endonuclease, or the nuclease domain thereof of embodiment 13, comprising up to 10 amino acid substitutions relative to the amino acid sequence of SEQ ID NO: 117.
Embodiment 15. A recombinant Clo051 endonuclease, or the nuclease domain thereof, comprising: an amino substitution at E99, F42, or a combination thereof relative to the amino acid sequence of SEQ ID NO: 118.
Embodiment 16. The recombinant Clo051 endonuclease, or the nuclease domain thereof of embodiment 15, comprising up to 10 amino acid substitutions relative to the amino acid sequence of SEQ ID NO: 118.
Embodiment 17. The recombinant Clo051 endonuclease or the nuclease domain thereof of any one of embodiments 1-16, wherein the Clo051 endonuclease, or the nuclease domain thereof comprises an amino substitution at E101 and wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.
Embodiment 18. The recombinant Clo051 endonuclease or the nuclease domain thereof of any one of embodiments 1-17, wherein the amino substitution at E101 is E101R, E101Q or E101K.
Embodiment 19. The recombinant Clo051 endonuclease or the nuclease domain thereof of any one of embodiments 1-18, wherein the amino substitution at E101 is E101R.
Embodiment 20. The recombinant Clo051 endonuclease or the nuclease domain thereof of any one of embodiments 1-19, wherein the Clo051 endonuclease, or the nuclease domain thereof comprises an amino substitution at F44 and wherein the amino substitution at F44 is F44S, F44T or F44A.
Embodiment 21. The recombinant Clo051 endonuclease or the nuclease domain thereof of any one of embodiments 1-20, wherein the amino substitution at F44 is F44T.
Embodiment 22. The recombinant Clo051 endonuclease or the nuclease domain thereof of any one of embodiments 1-21, wherein the Clo051 endonuclease or the nuclease domain thereof comprises an amino acid sequence of any one of SEQ ID NOS: 72-90.
Embodiment 23. The recombinant Clo051 endonuclease or the nuclease domain thereof of any one of embodiments 1-22, wherein the Clo051 endonuclease or the nuclease domain thereof comprises an amino acid sequence of any one of SEQ ID NOs: 84, 85 and 87.
Embodiment 24. The recombinant Clo051 endonuclease or the nuclease domain thereof of any one of embodiments 1-23, wherein the Clo051 endonuclease or the nuclease domain thereof comprises the amino acid sequence of SEQ ID NO: 85.
Embodiment 25. The recombinant Clo051 endonuclease or the nuclease domain thereof of any one of embodiments 1-24, wherein the Clo051 endonuclease or the nuclease domain thereof is encoded by a nucleic acid sequence with at least 90% identity to any one of SEQ ID NOs: 92-110.
Embodiment 26. The recombinant Clo051 endonuclease or the nuclease domain thereof of embodiment 25, wherein the Clo051 endonuclease or the nuclease domain thereof is encoded by a nucleic acid sequence with at least 90% identity to any one of SEQ ID NOS: 104, 105 and 107.
Embodiment 27. The recombinant Clo051 endonuclease or the nuclease domain thereof of embodiment 25 or embodiment 26, wherein the Clo051 endonuclease or the nuclease domain thereof is encoded by a nucleic acid sequence with at least 90% identity to SEQ ID NO: 105.
Embodiment 28. A fusion protein, comprising: (i) a DNA localization component, and (ii) the Clo051 endonuclease or the nuclease domain thereof of any one of embodiments 1-27.
Embodiment 29. The fusion protein of embodiment 28, wherein the DNA localization component comprises a DNA binding domain of a transcription activator-like effector (TALE).
Embodiment 30. The fusion protein of embodiment 29, wherein the DNA binding domain is a Xanthomonas TALE DNA binding domain or a Ralstonia TALE DNA binding domain.
Embodiment 31. The fusion protein of embodiment 28, wherein the DNA localization component comprises a catalytically inactive Cas protein, or a DNA binding domain thereof.
Embodiment 32. The fusion protein of embodiment 31, wherein the catalytically inactive Cas protein is a catalytically inactive Cas9 (dCas9), or a catalytically inactive small Cas9 (dSaCas9).
Embodiment 33. The fusion protein of embodiment 32, wherein the catalytically inactive Cas protein is a catalytically inactive Cas9 (dCas9) and wherein the dCas9 comprises the amino acid sequence of SEQ ID NO: 1.
Embodiment 34. The fusion protein of embodiment 32, wherein the catalytically inactive Cas protein is a catalytically inactive small Cas9 (dSaCas9) and wherein the dSaCas9 comprises the amino acid sequence of SEQ ID NO: 112.
Embodiment 35. A fusion protein, comprising: (i) a catalytically inactive Cas9 (dCas9), or an inactivated nuclease domain thereof, and (ii) a Clo051 endonuclease, or a nuclease domain thereof, wherein the Clo051 endonuclease or the nuclease domain thereof comprises (i) an amino substitution at E101 of SEQ ID NO: 23, or at the corresponding amino acid residue of SEQ ID NO: 71, SEQ ID NO: 117 or SEQ ID NO: 118, (ii) an amino substitution at F44 of SEQ ID NO: 23, or at the corresponding amino acid residue of SEQ ID NO: 71, SEQ ID NO: 117 or SEQ ID NO: 118, or (iii) a combination thereof.
Embodiment 36. The fusion protein of embodiment 35, wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.
Embodiment 37. The fusion protein of embodiment 35 or embodiment 36, wherein the amino substitution at E101 is E101R, E101Q or E101K.
Embodiment 38. The fusion protein of embodiment 37, wherein the amino substitution at E101 is E101R.
Embodiment 39. The fusion protein of any one of embodiments 35-38, wherein the amino substitution at F44 is F44S, F44T or F44A.
Embodiment 40. The fusion protein of any one of embodiments 28-39, wherein the fusion protein comprises an amino acid sequence of any one of SEQ ID NOS: 26-47.
Embodiment 41. The fusion protein of embodiment 40, wherein the fusion protein comprises an amino acid sequence of any one of SEQ ID NOS: 41, 42 or 44.
Embodiment 42. The fusion protein of embodiment 41, wherein the fusion protein comprises the amino acid sequence of SEQ ID NO: 42.
Embodiment 43. The fusion protein of any one of embodiments 28-42, wherein the fusion protein is encoded by a nucleic acid sequence with at least 90% identity to any one of SEQ ID NOS: 49-70.
Embodiment 44. The fusion protein of embodiment 43, wherein the fusion protein is encoded by a nucleic acid sequence with at least 90% identity to any one of SEQ ID NOS: 64, 65, and 67.
Embodiment 45. The fusion protein of embodiment 44, wherein the fusion protein is encoded by a nucleic acid sequence with at least 90% identity to SEQ ID NO: 65.
Embodiment 46. The fusion protein of any one of embodiments 28-45, wherein the fusion protein comprises a linker between the catalytically inactive Cas9 (dCas9), or the inactivated nuclease domain thereof, and the Clo051 endonuclease, or the nuclease domain thereof.
Embodiment 47. The fusion of embodiment 46, wherein the linker is a peptide linker.
Embodiment 48. The fusion protein of embodiment 46 or embodiment 47, wherein the peptide linker comprises the amino acid sequence of Gly-Gly-Gly-Gly-Ser (SEQ ID NO: 113).
Embodiment 49. The fusion protein of any one of embodiments 28-48, wherein the fusion protein recognizes a protospacer adjacent motif (PAM) sequence on a target double stranded nucleic acid.
Embodiment 50. The fusion protein of any one of embodiments 28-49, wherein the catalytically inactive Cas9 (dCas9) lacks a C-terminal SV40 nuclear localization sequence (NLS).
Embodiment 51. The fusion protein of embodiment 50, wherein the dCas9 lacking a C-terminal SV40 nuclear localization sequence (NLS) comprises the amino acid sequence of SEQ ID NO: 114.
Embodiment 52. A composition, comprising: (a) a left guide RNA (gRNA) and a right gRNA; and (b) the fusion protein of any one of embodiments 28-51.
Embodiment 53. A composition, comprising: (a) a left guide RNA (gRNA) and a right gRNA; and (b) a fusion protein, comprising: a catalytically inactive Cas9 (dCas9), and a Clo051 endonuclease or a nuclease domain thereof, wherein the Clo051 endonuclease or the nuclease domain thereof comprises an amino substitution at E101 of SEQ ID NO: 23, or at the corresponding amino acid residue of SEQ ID NO: 71, SEQ ID NO: 117 or SEQ ID NO: 118.
Embodiment 54. The composition of embodiment 53, wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.
Embodiment 55. The composition of any one of embodiments 52-54, wherein the 5′ end of the left gRNA and/or the 5′ end of the right gRNA are conjugated to a tRNA linker.
Embodiment 56. A composition, comprising: (a) a left guide RNA (gRNA) and a right gRNA, wherein the 5′ end of the left gRNA and the 5′ end of the right gRNA are conjugated to a tRNA linker; and (b) a fusion protein, comprising: (i) a catalytically inactive Cas9 (dCas9), wherein the dCas9 lacks a C-terminal SV40 nuclear localization sequence (NLS), and (ii) a Clo051 endonuclease or a nuclease domain thereof, wherein the Clo051 endonuclease or the nuclease domain thereof comprises an amino substitution at E101 of SEQ ID NO: 23, or at the corresponding amino acid residue of SEQ ID NO: 71, SEQ ID NO: 117 or SEQ ID NO: 118.
Embodiment 57. The composition of embodiment 55 or embodiment 56, wherein the tRNA linker comprises a nucleic acid sequence of SEQ ID NO: 111.
Embodiment 58. The composition of any one of embodiments 52-57, wherein the left gRNA and the fusion protein forms a left protein complex; and the right gRNA and the fusion protein form a right protein complex.
Embodiment 59. The composition of embodiment 58, wherein the Clo051 endonuclease or the nuclease domain thereof dimerizes resulting in a heterodimer of the left protein complex and the right protein complex.
Embodiment 60. The composition of any one of embodiments 52-59, wherein the left gRNA binds to one strand of a target double stranded nucleic acid adjacent to a left protospacer adjacent motif (PAM) sequence, and the right gRNA binds to the other strand of the target double stranded nucleic acid adjacent to a right protospacer adjacent motif (PAM) sequence. Embodiment 61. The composition of embodiment 60, wherein the fusion protein recognizes the left PAM sequence and the right PAM sequence on the target double stranded nucleic acid.
Embodiment 62. The composition of any one of embodiments 52-61, wherein the composition catalyzes a double stranded break in the target nucleic acid.
Embodiment 63. The composition of embodiment 62, wherein the double stranded break is located between the left PAM sequence and the right PAM sequence on the target double stranded nucleic acid.
Embodiment 64. A method of introducing a double stranded break in a target nucleic acid, the method comprising: bringing the composition of any one of embodiments 52-63 in contact with the target nucleic acid.
Embodiment 65. The method of embodiment 64, wherein the cutting efficiency of the composition is higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.
Embodiment 66. The method of embodiment 64, wherein the cutting efficiency of the composition is higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof.
Embodiment 67. The method of embodiment 65 or 66, wherein the cutting efficiency is measured using the ADE2 reporter assay.
Embodiment 68. The method of any one of embodiments 65-67, wherein the cutting efficiency of the composition is more than about 80%.
Embodiment 69. The method of any one of embodiments 64-68, wherein the contacting occurs in vitro, in vivo, or ex vivo.
Embodiment 70. The method of any one of embodiments 64-69, wherein the contacting occurs within a cell.
Embodiment 71. The method of embodiment 70, wherein the cell is a microbial cell, a fungal cell, a plant cell, or an animal cell.
Embodiment 72. The method of embodiment 71, wherein the animal cell is a mammalian cell.
Embodiment 73. The method of embodiment 71, wherein the microbial cell is a bacterial cell.
Embodiment 74. The method of embodiment 71, wherein the fungal cell is a yeast cell.
Embodiment 75. The method of any one of embodiments 64-74, wherein the cellular toxicity of the composition is lower than, or the same as, a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.
Embodiment 76. The method of any one of embodiments 64-74, wherein the cellular toxicity of the composition is lower than, or the same as, a control composition comprising: (a) a left gRNA and a right gRNA, wherein the left gRNA and right gRNA are conjugated to a RNA linker, and (b) a control fusion protein, comprising a catalytically inactive Cas9 (dCas9) and a wild type Clo051 endonuclease, or a nuclease domain thereof.
Embodiment 77. The method of embodiment 75 or 76, wherein the cellular toxicity is measured using the ADE2 reporter assay.
Embodiment 78. The method of embodiment 65, 66, 75 or 76, wherein the wild type Clo051 endonuclease or a nuclease domain thereof comprises an amino acid sequence of SEQ ID NO. 117 or 71.
Embodiment 79. A method of modifying a target double stranded nucleic acid, comprising: bringing (a) the composition of any one of embodiments 52-63 and (b) a donor nucleic acid, in contact with the target nucleic acid, wherein the donor nucleic acid is capable of homologous recombination with the target nucleic acid.
Embodiment 80. The method of embodiment 79, wherein the donor nucleic acid is integrated into the target nucleic acid through homologous recombination.
Embodiment 81. The method of embodiment 79 or embodiment 80, wherein the integration of the donor nucleic acid: (i) replaces one or more coding or non-coding sequences in the target nucleic acid, (ii) introduces one or more nucleotide mutations into the target nucleic acid, (iii) introduces a premature stop codon into the target nucleic acid, (iii) disrupts or introduces a splicing site in the target nucleic acid, or (vi) any combination thereof.
Embodiment 82. The method of any one of embodiments 79-81, wherein the contacting occurs in vitro, in vivo, or ex vivo.
Embodiment 83. The method of embodiment 82, wherein the contacting occurs in vivo, and the composition and the donor nucleic acid are administered to a subject, in need thereof.
Embodiment 84. The method of embodiment 83, wherein the subject is a human subject.

What is claimed is:

1. A recombinant Clo051 endonuclease, or a nuclease domain thereof, comprising:
(i) an amino substitution at E101 of SEQ ID NO: 23, or at a corresponding amino acid residue, of a wild type Clo051 endonuclease or a nuclease domain thereof,
(ii) an amino substitution at F44 of SEQ ID NO: 23, or at a corresponding amino acid residue, of a wild type Clo051 endonuclease or a nuclease domain thereof, or
(iii) a combination thereof.

2. A recombinant Clo051 endonuclease, or a nuclease domain thereof, comprising:
(i) an amino substitution at E101 of SEQ ID NO: 23, or at the corresponding amino acid residue of SEQ ID NO: 71, SEQ ID NO: 117 or SEQ ID NO: 118,
(ii) an amino substitution at F44 of SEQ ID NO: 23, or at the corresponding amino acid residue of SEQ ID NO: 71, SEQ ID NO: 117 or SEQ ID NO: 118, or
(iii) a combination thereof.

3. The recombinant Clo051 endonuclease, or the nuclease domain thereof of claim 1, wherein the recombinant Clo051 endonuclease, or the nuclease domain thereof comprises up to 10 amino acid substitutions relative to SEQ ID NO: 23 or the amino acid sequence of a wild type Clo051 endonuclease or a nuclease domain thereof.

4. The recombinant Clo051 endonuclease, or the nuclease domain thereof of claim 2, wherein the recombinant Clo051 endonuclease, or the nuclease domain thereof comprises up to 10 amino acid substitutions relative to SEQ ID NO: 23, SEQ ID NO: 71, SEQ ID NO: 117 or SEQ ID NO: 118.

5. The recombinant Clo051 endonuclease, or the nuclease domain thereof of any one of claims 1-4, comprising: an amino substitution at E90, F33, or a combination thereof relative to the amino acid sequence of SEQ ID NO: 71.

6. The recombinant Clo051 endonuclease, or the nuclease domain thereof of any one of claims 1-4, comprising: an amino substitution at E101, F44, or a combination thereof relative to the amino acid sequence of SEQ ID NO: 23.

7. The recombinant Clo051 endonuclease, or the nuclease domain thereof of any one of claims 1-4, comprising: an amino substitution at E478, F421, or a combination thereof relative to the amino acid sequence of SEQ ID NO: 117.

8. The recombinant Clo051 endonuclease, or the nuclease domain thereof of any one of claims 1-4, comprising: an amino substitution at E99, F42, or a combination thereof relative to the amino acid sequence of SEQ ID NO: 118.

9. A recombinant Clo051 endonuclease, or the nuclease domain thereof, comprising: an amino substitution at E90, F33, or a combination thereof relative to the amino acid sequence of SEQ ID NO: 71.

10. The recombinant Clo051 endonuclease, or the nuclease domain thereof of claim 9, comprising up to 10 amino acid substitutions relative to the amino acid sequence of SEQ ID NO: 71.

11. A recombinant Clo051 endonuclease, or the nuclease domain thereof, comprising: an amino substitution at E101, F44, or a combination thereof relative to the amino acid sequence of SEQ ID NO: 23.

12. The recombinant Clo051 endonuclease, or the nuclease domain thereof of claim 11, comprising up to 10 amino acid substitutions relative to the amino acid sequence of SEQ ID NO: 23.

13. A recombinant Clo051 endonuclease, or the nuclease domain thereof, comprising: an amino substitution at E478, F421, or a combination thereof relative to the amino acid sequence of SEQ ID NO: 117.

14. The recombinant Clo051 endonuclease, or the nuclease domain thereof of claim 13, comprising up to 10 amino acid substitutions relative to the amino acid sequence of SEQ ID NO: 117.

15. A recombinant Clo051 endonuclease, or the nuclease domain thereof, comprising: an amino substitution at E99, F42, or a combination thereof relative to the amino acid sequence of SEQ ID NO: 118.

16. The recombinant Clo051 endonuclease, or the nuclease domain thereof of claim 15, comprising up to 10 amino acid substitutions relative to the amino acid sequence of SEQ ID NO: 118.

17. The recombinant Clo051 endonuclease or the nuclease domain thereof of any one of claims 1-16, wherein the Clo051 endonuclease, or the nuclease domain thereof comprises an amino substitution at E101 and wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.

18. The recombinant Clo051 endonuclease or the nuclease domain thereof of any one of claims 1-17, wherein the amino substitution at E101 is E101R, E101Q or E101K.

19. The recombinant Clo051 endonuclease or the nuclease domain thereof of any one of claims 1-18, wherein the amino substitution at E101 is E101R.

20. The recombinant Clo051 endonuclease or the nuclease domain thereof of any one of claims 1-19, wherein the Clo051 endonuclease, or the nuclease domain thereof comprises an amino substitution at F44 and wherein the amino substitution at F44 is F44S, F44T or F44A.

21. The recombinant Clo051 endonuclease or the nuclease domain thereof of any one of claims 1-20, wherein the amino substitution at F44 is F44T.

22. The recombinant Clo051 endonuclease or the nuclease domain thereof of any one of claims 1-21, wherein the Clo051 endonuclease or the nuclease domain thereof comprises an amino acid sequence of any one of SEQ ID NOS: 72-90.

23. The recombinant Clo051 endonuclease or the nuclease domain thereof of any one of claims 1-22, wherein the Clo051 endonuclease or the nuclease domain thereof comprises an amino acid sequence of any one of SEQ ID NOs: 84, 85 and 87.

24. The recombinant Clo051 endonuclease or the nuclease domain thereof of any one of claims 1-23, wherein the Clo051 endonuclease or the nuclease domain thereof comprises the amino acid sequence of SEQ ID NO: 85.

25. The recombinant Clo051 endonuclease or the nuclease domain thereof of any one of claims 1-24, wherein the Clo051 endonuclease or the nuclease domain thereof is encoded by a nucleic acid sequence with at least 90% identity to any one of SEQ ID NOs: 92-110.

26. The recombinant Clo051 endonuclease or the nuclease domain thereof of claim 25, wherein the Clo051 endonuclease or the nuclease domain thereof is encoded by a nucleic acid sequence with at least 90% identity to any one of SEQ ID NOs: 104, 105 and 107.

27. The recombinant Clo051 endonuclease or the nuclease domain thereof of claim 25 or claim 26, wherein the Clo051 endonuclease or the nuclease domain thereof is encoded by a nucleic acid sequence with at least 90% identity to SEQ ID NO: 105.

28. A fusion protein, comprising: (i) a DNA localization component, and (ii) the Clo051 endonuclease or the nuclease domain thereof of any one of claims 1-27.

29. The fusion protein of claim 28, wherein the DNA localization component comprises a DNA binding domain of a transcription activator-like effector (TALE).

30. The fusion protein of claim 29, wherein the DNA binding domain is a Xanthomonas TALE DNA binding domain or a Ralstonia TALE DNA binding domain.

31. The fusion protein of claim 28, wherein the DNA localization component comprises a catalytically inactive Cas protein, or a DNA binding domain thereof.

32. The fusion protein of claim 31, wherein the catalytically inactive Cas protein is a catalytically inactive Cas9 (dCas9), or a catalytically inactive small Cas9 (dSaCas9).

33. The fusion protein of claim 32, wherein the catalytically inactive Cas protein is a catalytically inactive Cas9 (dCas9) and wherein the dCas9 comprises the amino acid sequence of SEQ ID NO: 1.

34. The fusion protein of claim 32, wherein the catalytically inactive Cas protein is a catalytically inactive small Cas9 (dSaCas9) and wherein the dSaCas9 comprises the amino acid sequence of SEQ ID NO: 112.

35. A fusion protein, comprising: (i) a catalytically inactive Cas9 (dCas9), or an inactivated nuclease domain thereof, and (ii) a Clo051 endonuclease, or a nuclease domain thereof, wherein the Clo051 endonuclease or the nuclease domain thereof comprises (i) an amino substitution at E101 of SEQ ID NO: 23, or at the corresponding amino acid residue of SEQ ID NO: 71, SEQ ID NO: 117 or SEQ ID NO: 118, (ii) an amino substitution at F44 of SEQ ID NO: 23, or at the corresponding amino acid residue of SEQ ID NO: 71, SEQ ID NO: 117 or SEQ ID NO: 118, or (iii) a combination thereof.

36. The fusion protein of claim 35, wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.

37. The fusion protein of claim 35 or claim 36, wherein the amino substitution at E101 is E101R, E101Q or E101K.

38. The fusion protein of claim 37, wherein the amino substitution at E101 is E101R.

39. The fusion protein of any one of claims 35-38, wherein the amino substitution at F44 is F44S, F44T or F44A.

40. The fusion protein of any one of claims 28-39, wherein the fusion protein comprises an amino acid sequence of any one of SEQ ID NOS: 26-47.

41. The fusion protein of claim 40, wherein the fusion protein comprises an amino acid sequence of any one of SEQ ID NOS: 41, 42 or 44.

42. The fusion protein of claim 41, wherein the fusion protein comprises the amino acid sequence of SEQ ID NO: 42.

43. The fusion protein of any one of claims 28-42, wherein the fusion protein is encoded by a nucleic acid sequence with at least 90% identity to any one of SEQ ID NOS: 49-70.

44. The fusion protein of claim 43, wherein the fusion protein is encoded by a nucleic acid sequence with at least 90% identity to any one of SEQ ID NOS: 64, 65, and 67.

45. The fusion protein of claim 44, wherein the fusion protein is encoded by a nucleic acid sequence with at least 90% identity to SEQ ID NO: 65.

46. The fusion protein of any one of claims 28-45, wherein the fusion protein comprises a linker between the catalytically inactive Cas9 (dCas9), or the inactivated nuclease domain thereof, and the Clo051 endonuclease, or the nuclease domain thereof.

47. The fusion of claim 46, wherein the linker is a peptide linker.

48. The fusion protein of claim 46 or claim 47, wherein the peptide linker comprises the amino acid sequence of Gly-Gly-Gly-Gly-Ser (SEQ ID NO: 113).

49. The fusion protein of any one of claims 28-48, wherein the fusion protein recognizes a protospacer adjacent motif (PAM) sequence on a target double stranded nucleic acid.

50. The fusion protein of any one of claims 28-49, wherein the catalytically inactive Cas9 (dCas9) lacks a C-terminal SV40 nuclear localization sequence (NLS).

51. The fusion protein of claim 50, wherein the dCas9 lacking a C-terminal SV40 nuclear localization sequence (NLS) comprises the amino acid sequence of SEQ ID NO: 114.

52. A composition, comprising: (a) a left guide RNA (gRNA) and a right gRNA; and (b) the fusion protein of any one of claims 28-51.

53. A composition, comprising: (a) a left guide RNA (gRNA) and a right gRNA; and (b) a fusion protein, comprising: a catalytically inactive Cas9 (dCas9), and a Clo051 endonuclease or a nuclease domain thereof, wherein the Clo051 endonuclease or the nuclease domain thereof comprises an amino substitution at E101 of SEQ ID NO: 23, or at the corresponding amino acid residue of SEQ ID NO: 71, SEQ ID NO: 117 or SEQ ID NO: 118.

54. The composition of claim 53, wherein the amino substitution at E101 is E101S, E101N E101A, E101L, E101I, E101G, E101T, E101F, E101Y, E101W, E101P, E101H, E101Q, E101R, E101M, E101K, E101V, E101D, or E101C.

55. The composition of any one of claims 52-54, wherein the 5′ end of the left gRNA and/or the 5′ end of the right gRNA are conjugated to a tRNA linker.

56. A composition, comprising: (a) a left guide RNA (gRNA) and a right gRNA, wherein the 5′ end of the left gRNA and the 5′ end of the right gRNA are conjugated to a tRNA linker; and (b) a fusion protein, comprising: (i) a catalytically inactive Cas9 (dCas9), wherein the dCas9 lacks a C-terminal SV40 nuclear localization sequence (NLS), and (ii) a Clo051 endonuclease or a nuclease domain thereof, wherein the Clo051 endonuclease or the nuclease domain thereof comprises an amino substitution at E101 of SEQ ID NO: 23, or at the corresponding amino acid residue of SEQ ID NO: 71, SEQ ID NO: 117 or SEQ ID NO: 118.

57. The composition of claim 55 or claim 56, wherein the tRNA linker comprises a nucleic acid sequence of SEQ ID NO: 111.

58. The composition of any one of claims 52-57, wherein the left gRNA and the fusion protein forms a left protein complex; and the right gRNA and the fusion protein form a right protein complex.

59. The composition of claim 58, wherein the Clo051 endonuclease or the nuclease domain thereof dimerizes resulting in a heterodimer of the left protein complex and the right protein complex.

60. The composition of any one of claims 52-59, wherein the left gRNA binds to one strand of a target double stranded nucleic acid adjacent to a left protospacer adjacent motif (PAM) sequence, and the right gRNA binds to the other strand of the target double stranded nucleic acid adjacent to a right protospacer adjacent motif (PAM) sequence.

61. The composition of claim 60, wherein the fusion protein recognizes the left PAM sequence and the right PAM sequence on the target double stranded nucleic acid.

62. The composition of any one of claims 52-61, wherein the composition catalyzes a double stranded break in the target nucleic acid.

63. The composition of claim 62, wherein the double stranded break is located between the left PAM sequence and the right PAM sequence on the target double stranded nucleic acid.

64. A method of introducing a double stranded break in a target nucleic acid, the method comprising: bringing the composition of any one of claims 52-63 in contact with the target nucleic acid.

65. The method of claim 64, wherein the cutting efficiency of the composition is higher than a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.

66. The method of claim 64, wherein the cutting efficiency of the composition is higher than a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease or a nuclease domain thereof.

67. The method of claim 65 or 66, wherein the cutting efficiency is measured using the ADE2 reporter assay.

68. The method of any one of claims 65-67, wherein the cutting efficiency of the composition is more than about 80%.

69. The method of any one of claims 64-68, wherein the contacting occurs in vitro, in vivo, or ex vivo.

70. The method of any one of claims 64-69, wherein the contacting occurs within a cell.

71. The method of claim 70, wherein the cell is a microbial cell, a fungal cell, a plant cell, or an animal cell.

72. The method of claim 71, wherein the animal cell is a mammalian cell.

73. The method of claim 71, wherein the microbial cell is a bacterial cell.

74. The method of claim 71, wherein the fungal cell is a yeast cell.

75. The method of any one of claims 64-74, wherein the cellular toxicity of the composition is lower than, or the same as, a control composition comprising: (a) a left gRNA and a right gRNA, wherein the left gRNA and right gRNA are not conjugated to a tRNA linker, and (b) a control fusion protein, comprising a catalytically inactive Cas9 (dCas9) and a wild type Clo051 endonuclease or a nuclease domain thereof.

76. The method of any one of claims 64-74, wherein the cellular toxicity of the composition is lower than, or the same as, a control composition comprising: (a) a left gRNA and a right gRNA, wherein the left gRNA and right gRNA are conjugated to a tRNA linker, and (b) a control fusion protein, comprising a catalytically inactive Cas9 (dCas9) and a wild type Clo051 endonuclease, or a nuclease domain thereof.

77. The method of claim 75 or 76, wherein the cellular toxicity is measured using the ADE2 reporter assay.

78. The method of claim 65, 66, 75 or 76, wherein the wild type Clo051 endonuclease or a nuclease domain thereof comprises an amino acid sequence of SEQ ID NO. 117 or 71.

79. A method of modifying a target double stranded nucleic acid, comprising: bringing (a) the composition of any one of claims 52-63 and (b) a donor nucleic acid, in contact with the target nucleic acid, wherein the donor nucleic acid is capable of homologous recombination with the target nucleic acid.

80. The method of claim 79, wherein the donor nucleic acid is integrated into the target nucleic acid through homologous recombination.

81. The method of claim 79 or claim 80, wherein the integration of the donor nucleic acid:
(i) replaces one or more coding or non-coding sequences in the target nucleic acid, (ii) introduces one or more nucleotide mutations into the target nucleic acid, (iii) introduces a premature stop codon into the target nucleic acid, (iii) disrupts or introduces a splicing site in the target nucleic acid, or (vi) any combination thereof.

82. The method of any one of claims 79-81, wherein the contacting occurs in vitro, in vivo, or ex vivo.

83. The method of claim 82, wherein the contacting occurs in vivo, and the composition and the donor nucleic acid are administered to a subject, in need thereof.

84. The method of claim 83, wherein the subject is a human subject.


SEQUENCE LISTING (140 sequences)

SEQ ID NO: 1 - protein, 1377 aa, synthetic construct
  REGION 1..1377: note = dCas9 with C-terminal NLS
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 2 - DNA, 4134 bp, synthetic construct
  misc_feature 1..4134: note = dCas9 with C-terminal NLS
  source 1..4134: mol_type = other DNA
atggacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 3 - protein, 1591 aa, synthetic construct
  REGION 1..1591: note = WT dCas9-Clo51 (with N- and C-terminal NLS)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 4 - DNA, 4776 bp, synthetic construct
  misc_feature 1..4776: note = WT dCas9-Clo51 (with N- and C-terminal NLS)
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 5 - protein, 1591 aa, synthetic construct
  REGION 1..1591: note = dCas9-Clo51 F44S
MAPKKKRKVGSEGIKSNISLLKDELRGQISHISHEYLSLIDLASDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 6 - protein, 1591 aa, synthetic construct
  REGION 1..1591: note = dCas9-Clo51 F44T
MAPKKKRKVGSEGIKSNISLLKDELRGQISHISHEYLSLIDLATDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 7 - protein, 1591 aa, synthetic construct
  REGION 1..1591: note = dCas9-Clo51 F44A
MAPKKKRKVGSEGIKSNISLLKDELRGQISHISHEYLSLIDLAADSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 8 - protein, 1591 aa, synthetic construct
  REGION 1..1591: note = dCas9-Clo51 E101S
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSSGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 9 - protein, 1591 aa, synthetic construct
  REGION 1..1591: note = dCas9-Clo51 E101N
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSNGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 10 - protein, 1591 aa, synthetic construct
  REGION 1..1591: note = dCas9-Clo51 E101A
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSAGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 11 - protein, 1591 aa, synthetic construct
  REGION 1..1591: note = dCas9-Clo51 E101Q
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSQGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 12 - protein, 1591 aa, synthetic construct
  REGION 1..1591: note = dCas9-Clo51 E101R
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSRGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 13 - protein, 1591 aa, synthetic construct
  REGION 1..1591: note = dCas9-Clo51 E101K
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSKGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 14 - DNA, 4776 bp, synthetic construct
  misc_feature 1..4776: note = dCas9-Clo51 F44S
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccagcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 15 - DNA, 4776 bp, synthetic construct
  misc_feature 1..4776: note = dCas9-Clo51 F44T
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccaccgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 16 - DNA, 4776 bp, synthetic construct
  misc_feature 1..4776: note = dCas9-Clo51 F44A
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccgccgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 17 - DNA, 4776 bp, synthetic construct
  misc_feature 1..4776: note = dCas9-Clo51 E101S
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 18 - DNA, 4776 bp, synthetic construct
  misc_feature 1..4776: note = dCas9-Clo51 E101N
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 19 - DNA, 4776 bp, synthetic construct
  misc_feature 1..4776: note = dCas9-Clo51 E101A
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 20 - DNA, 4776 bp, synthetic construct
  misc_feature 1..4776: note = dCas9-Clo51 E101Q
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctcaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 21 - DNA, 4776 bp, synthetic construct
  misc_feature 1..4776: note = dCas9-Clo51 E101R
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctagaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 22 - DNA, 4776 bp, synthetic construct
  misc_feature 1..4776: note = dCas9-Clo51 E101K
  source 1..4776: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatccccaaagaaaaagcgtaaagtctag

SEQ ID NO: 23 - protein, 210 aa, synthetic construct
  REGION 1..210: note = Wildtype Clo51 w/ N-terminus SV40 nuclear localization sequence (NLS)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 24 - DNA, 630 bp, synthetic construct
  misc_feature 1..630: note = Wildtype Clo51 w/ N-terminus SV40 nuclear localization sequence (NLS)
  source 1..630: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 25 - protein, 1584 aa, synthetic construct
  REGION 1..1584: note = Wildtype dCas9-Clo51 w/o C-terminus SV40 NLS (WT dCas9-Clo51_dC)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 26 - protein, 1584 aa, synthetic construct
  REGION 1..1584: note = dCas9-Clo51 F44S w/o C-terminus SV40 NLS (dCas9-Clo51_dC F44S)
MAPKKKRKVGSEGIKSNISLLKDELRGQISHISHEYLSLIDLASDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 27 - protein, 1584 aa, synthetic construct
  REGION 1..1584: note = dCas9-Clo51 F44T w/o C-terminus SV40 NLS (dCas9-Clo51_dC F44T)
MAPKKKRKVGSEGIKSNISLLKDELRGQISHISHEYLSLIDLATDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 28 - protein, 1584 aa, synthetic construct
  REGION 1..1584: note = dCas9-Clo51 F44A w/o C-terminus SV40 NLS (dCas9-Clo51_dC F44A)
MAPKKKRKVGSEGIKSNISLLKDELRGQISHISHEYLSLIDLAADSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 29 - protein, 1584 aa, synthetic construct
  REGION 1..1584: note = dCas9-Clo51 E101S w/o C-terminus SV40 NLS (dCas9-Clo51_dC E101S)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSSGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 30 - protein, 1584 aa, synthetic construct
  REGION 1..1584: note = dCas9-Clo51 E101N w/o C-terminus SV40 NLS (dCas9-Clo51_dC E101N)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSNGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 31 - protein, 1584 aa, synthetic construct
  REGION 1..1584: note = dCas9-Clo51 E101A w/o C-terminus SV40 NLS (dCas9-Clo51_dC E101A)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSAGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 32 - protein, 1584 aa, synthetic construct
  REGION 1..1584: note = dCas9-Clo51 E101L w/o C-terminus SV40 NLS (dCas9-Clo51_dC E101L)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSLGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 33 - protein, 1584 aa, synthetic construct
  REGION 1..1584: note = dCas9-Clo51 E101I w/o C-terminus SV40 NLS (dCas9-Clo51_dC E101I)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSIGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 34 - protein, 1584 aa, synthetic construct
  REGION 1..1584: note = dCas9-Clo51 E101G w/o C-terminus SV40 NLS (dCas9-Clo51_dC E101G)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSGGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 35 - protein, 1584 aa, synthetic construct
  REGION 1..1584: note = dCas9-Clo51 E101T w/o C-terminus SV40 NLS (dCas9-Clo51_dC E101T)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSTGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 36 - protein, 1584 aa, synthetic construct
  REGION 1..1584: note = dCas9-Clo51 E101F w/o C-terminus SV40 NLS (dCas9-Clo51_dC E101F)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSFGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 37 - protein, 1584 aa, synthetic construct
  REGION 1..1584: note = dCas9-Clo51 E101Y w/o C-terminus SV40 NLS (dCas9-Clo51_dC E101Y)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSYGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 38 - protein, 1584 aa, synthetic construct
  REGION 1..1584: note = dCas9-Clo51 E101W w/o C-terminus SV40 NLS (dCas9-Clo51_dC E101W)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSWGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 39 - protein, 1584 aa, synthetic construct
  REGION 1..1584: note = dCas9-Clo51 E101P w/o C-terminus SV40 NLS (dCas9-Clo51_dC E101P)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSPGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 40 - protein, 1584 aa, synthetic construct
  REGION 1..1584: note = dCas9-Clo51 E101H w/o C-terminus SV40 NLS (dCas9-Clo51_dC E101H)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSHGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 41 - protein, 1584 aa, synthetic construct
  REGION 1..1584: note = dCas9-Clo51 E101Q w/o C-terminus SV40 NLS (dCas9-Clo51_dC E101Q)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSQGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 42 - protein, 1584 aa, synthetic construct
  REGION 1..1584: note = dCas9-Clo51 E101R w/o C-terminus SV40 NLS (dCas9-Clo51_dC E101R)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSRGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 43 - protein, 1584 aa, synthetic construct
  REGION 1..1584: note = dCas9-Clo51 E101M w/o C-terminus SV40 NLS (dCas9-Clo51_dC E101M)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSMGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 44 - protein, 1584 aa, synthetic construct
  REGION 1..1584: note = dCas9-Clo51 E101K w/o C-terminus SV40 NLS (dCas9-Clo51_dC E101K)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSKGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 45 - protein, 1584 aa, synthetic construct
  REGION 1..1584: note = dCas9-Clo51 E101V w/o C-terminus SV40 NLS (dCas9-Clo51_dC E101V)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSVGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 46 - protein, 1584 aa, synthetic construct
  REGION 1..1584: note = dCas9-Clo51 E101D w/o C-terminus SV40 NLS (dCas9-Clo51_dC E101D)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSDGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 47 - protein, 1584 aa, synthetic construct
  REGION 1..1584: note = dCas9-Clo51 E101C w/o C-terminus SV40 NLS (dCas9-Clo51_dC E101C)
MAPKKKRKVGSEGIKSNISLLKDELRGQISHISHEYLSLIDLAFDSKQNRLFEMKVLELLVNEYGFKGRHLGGSRKPDGIVYSTTLEDNFGIIVDTKAYSCGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGS

SEQ ID NO: 48 - DNA, 4755 bp, synthetic construct
  misc_feature 1..4755: note = Wildtype dCas9-Clo51 w/o C-terminus SV40 NLS (WT dCas9-Clo51_dC)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 49 - DNA, 4755 bp, synthetic construct
  misc_feature 1..4755: note = dCas9-Clo51 F44S w/o C-terminus SV40 NLS (dCas9-Clo51_dC F44S)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccagcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 50 - DNA, 4755 bp, synthetic construct
  misc_feature 1..4755: note = dCas9-Clo51 F44T w/o C-terminus SV40 NLS (dCas9-Clo51_dC F44T)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccaccgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 51 - DNA, 4755 bp, synthetic construct
  misc_feature 1..4755: note = dCas9-Clo51 F44A w/o C-terminus SV40 NLS (dCas9-Clo51_dC F44A)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccgccgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 52 - DNA, 4755 bp, synthetic construct
  misc_feature 1..4755: note = dCas9-Clo51 E101S w/o C-terminus SV40 NLS (dCas9-Clo51_dC E101S)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 53 - DNA, 4755 bp, synthetic construct
  misc_feature 1..4755: note = dCas9-Clo51 E101N w/o C-terminus SV40 NLS (dCas9-Clo51_dC E101N)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 54 - DNA, 4755 bp, synthetic construct
  misc_feature 1..4755: note = dCas9-Clo51 E101A w/o C-terminus SV40 NLS (dCas9-Clo51_dC E101A)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 55 - DNA, 4755 bp, synthetic construct
  misc_feature 1..4755: note = dCas9-Clo51 E101L w/o C-terminus SV40 NLS (dCas9-Clo51_dC E101L)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctctaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 56 - DNA, 4755 bp, synthetic construct
  misc_feature 1..4755: note = dCas9-Clo51 E101I w/o C-terminus SV40 NLS (dCas9-Clo51_dC E101I)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctataggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 57 - DNA, 4755 bp, synthetic construct
  misc_feature 1..4755: note = dCas9-Clo51 E101G w/o C-terminus SV40 NLS (dCas9-Clo51_dC E101G)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctggaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 58 - DNA, 4755 bp, synthetic construct
  misc_feature 1..4755: note = dCas9-Clo51 E101T w/o C-terminus SV40 NLS (dCas9-Clo51_dC E101T)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctacaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 59 - DNA, 4755 bp, synthetic construct
  misc_feature 1..4755: note = dCas9-Clo51 E101F w/o C-terminus SV40 NLS (dCas9-Clo51_dC E101F)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctttcggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 60 - DNA, 4755 bp, synthetic construct
  misc_feature 1..4755: note = dCas9-Clo51 E101Y w/o C-terminus SV40 NLS (dCas9-Clo51_dC E101Y)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 61 - DNA, 4755 bp, synthetic construct
  misc_feature 1..4755: note = dCas9-Clo51 E101W w/o C-terminus SV40 NLS (dCas9-Clo51_dC E101W)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttggggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 62 - DNA, 4755 bp, synthetic construct
  misc_feature 1..4755: note = dCas9-Clo51 E101P w/o C-terminus SV40 NLS (dCas9-Clo51_dC E101P)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctccaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 63 - DNA, 4755 bp, synthetic construct
  misc_feature 1..4755: note = dCas9-Clo51 E101H w/o C-terminus SV40 NLS (dCas9-Clo51_dC E101H)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctcacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 64 - DNA, 4755 bp, synthetic construct
  misc_feature 1..4755: note = dCas9-Clo51 E101Q w/o C-terminus SV40 NLS (dCas9-Clo51_dC E101Q)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctcaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 65 - DNA, 4755 bp, synthetic construct
  misc_feature 1..4755: note = dCas9-Clo51 E101R w/o C-terminus SV40 NLS (dCas9-Clo51_dC E101R)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctagaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 66 - DNA, 4755 bp, synthetic construct
  misc_feature 1..4755: note = dCas9-Clo51 E101M w/o C-terminus SV40 NLS (dCas9-Clo51_dC E101M)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctatgggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 67 - DNA, 4755 bp, synthetic construct
  misc_feature 1..4755: note = dCas9-Clo51 E101K w/o C-terminus SV40 NLS (dCas9-Clo51_dC E101K)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 68 - DNA, 4755 bp, synthetic construct
  misc_feature 1..4755: note = dCas9-Clo51 E101V w/o C-terminus SV40 NLS (dCas9-Clo51_dC E101V)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgtaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 69 - DNA, 4755 bp, synthetic construct
  misc_feature 1..4755: note = dCas9-Clo51 E101D w/o C-terminus SV40 NLS (dCas9-Clo51_dC E101D)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgatggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 70 - DNA, 4755 bp, synthetic construct
  misc_feature 1..4755: note = dCas9-Clo51 E101C w/o C-terminus SV40 NLS (dCas9-Clo51_dC E101C)
  source 1..4755: mol_type = other DNA
atggctccaaagaagaagagaaaggttggttctgaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttgcggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatacggtggtggtggttctgacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 71 - protein, 199 aa, synthetic construct
  REGION 1..199: note = Clo51 WT
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 72 - protein, 199 aa, synthetic construct
  REGION 1..199: note = Clo51 E101S
EGIKSNISLLKDELRGQISHISHEYLSLIDLAFDSKQNRLFEMKVLELLVNEYGFKGRHLGGSRKPDGIVYSTTLEDNFGIIVDTKAYSSGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 73 - protein, 199 aa, synthetic construct
  REGION 1..199: note = Clo51 E101N
EGIKSNISLLKDELRGQISHISHEYLSLIDLAFDSKQNRLFEMKVLELLVNEYGFKGRHLGGSRKPDGIVYSTTLEDNFGIIVDTKAYSNGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 74 - protein, 199 aa, synthetic construct
  REGION 1..199: note = Clo51 E101A
EGIKSNISLLKDELRGQISHISHEYLSLIDLAFDSKQNRLFEMKVLELLVNEYGFKGRHLGGSRKPDGIVYSTTLEDNFGIIVDTKAYSAGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 75 - protein, 199 aa, synthetic construct
  REGION 1..199: note = Clo51 E101L
EGIKSNISLLKDELRGQISHISHEYLSLIDLAFDSKQNRLFEMKVLELLVNEYGFKGRHLGGSRKPDGIVYSTTLEDNFGIIVDTKAYSLGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 76 - protein, 199 aa, synthetic construct
  REGION 1..199: note = Clo51 E101I
EGIKSNISLLKDELRGQISHISHEYLSLIDLAFDSKQNRLFEMKVLELLVNEYGFKGRHLGGSRKPDGIVYSTTLEDNFGIIVDTKAYSIGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 77 - protein, 199 aa, synthetic construct
  REGION 1..199: note = Clo51 E101G
EGIKSNISLLKDELRGQISHISHEYLSLIDLAFDSKQNRLFEMKVLELLVNEYGFKGRHLGGSRKPDGIVYSTTLEDNFGIIVDTKAYSGGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 78 - protein, 199 aa, synthetic construct
  REGION 1..199: note = Clo51 E101T
EGIKSNISLLKDELRGQISHISHEYLSLIDLAFDSKQNRLFEMKVLELLVNEYGFKGRHLGGSRKPDGIVYSTTLEDNFGIIVDTKAYSTGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 79 - protein, 199 aa, synthetic construct
  REGION 1..199: note = Clo51 E101F
EGIKSNISLLKDELRGQISHISHEYLSLIDLAFDSKQNRLFEMKVLELLVNEYGFKGRHLGGSRKPDGIVYSTTLEDNFGIIVDTKAYSFGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 80 - protein, 199 aa, synthetic construct
  REGION 1..199: note = Clo51 E101Y
EGIKSNISLLKDELRGQISHISHEYLSLIDLAFDSKQNRLFEMKVLELLVNEYGFKGRHLGGSRKPDGIVYSTTLEDNFGIIVDTKAYSYGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 81 - protein, 199 aa, synthetic construct
  REGION 1..199: note = Clo51 E101W
EGIKSNISLLKDELRGQISHISHEYLSLIDLAFDSKQNRLFEMKVLELLVNEYGFKGRHLGGSRKPDGIVYSTTLEDNFGIIVDTKAYSWGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 82 - protein, 199 aa, synthetic construct
  REGION 1..199: note = Clo51 E101P
EGIKSNISLLKDELRGQISHISHEYLSLIDLAFDSKQNRLFEMKVLELLVNEYGFKGRHLGGSRKPDGIVYSTTLEDNFGIIVDTKAYSPGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 83 - protein, 199 aa, synthetic construct
  REGION 1..199: note = Clo51 E101H
EGIKSNISLLKDELRGQISHISHEYLSLIDLAFDSKQNRLFEMKVLELLVNEYGFKGRHLGGSRKPDGIVYSTTLEDNFGIIVDTKAYSHGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 84 - protein, 199 aa, synthetic construct
  REGION 1..199: note = Clo51 E101Q
EGIKSNISLLKDELRGQISHISHEYLSLIDLAFDSKQNRLFEMKVLELLVNEYGFKGRHLGGSRKPDGIVYSTTLEDNFGIIVDTKAYSQGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 85 - protein, 199 aa, synthetic construct
  REGION 1..199: note = Clo51 E101R
EGIKSNISLLKDELRGQISHISHEYLSLIDLAFDSKQNRLFEMKVLELLVNEYGFKGRHLGGSRKPDGIVYSTTLEDNFGIIVDTKAYSRGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 86 - protein, 199 aa, synthetic construct
  REGION 1..199: note = Clo51 E101M
EGIKSNISLLKDELRGQISHISHEYLSLIDLAFDSKQNRLFEMKVLELLVNEYGFKGRHLGGSRKPDGIVYSTTLEDNFGIIVDTKAYSMGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 87 - protein, 199 aa, synthetic construct
  REGION 1..199: note = Clo51 E101K
EGIKSNISLLKDELRGQISHISHEYLSLIDLAFDSKQNRLFEMKVLELLVNEYGFKGRHLGGSRKPDGIVYSTTLEDNFGIIVDTKAYSKGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 88 - protein, 199 aa, synthetic construct
  REGION 1..199: note = Clo51 E101V
EGIKSNISLLKDELRGQISHISHEYLSLIDLAFDSKQNRLFEMKVLELLVNEYGFKGRHLGGSRKPDGIVYSTTLEDNFGIIVDTKAYSVGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 89 - protein, 199 aa, synthetic construct
  REGION 1..199: note = Clo51 E101D
EGIKSNISLLKDELRGQISHISHEYLSLIDLAFDSKQNRLFEMKVLELLVNEYGFKGRHLGGSRKPDGIVYSTTLEDNFGIIVDTKAYSDGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 90 - protein, 199 aa, synthetic construct
  REGION 1..199: note = Clo51 E101C
EGIKSNISLLKDELRGQISHISHEYLSLIDLAFDSKQNRLFEMKVLELLVNEYGFKGRHLGGSRKPDGIVYSTTLEDNFGIIVDTKAYSCGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 91 - DNA, 597 bp, synthetic construct
  misc_feature 1..597: note = Clo51 WT
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 92 - DNA, 597 bp, synthetic construct
  misc_feature 1..597: note = Clo51 E101S
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 93 - DNA, 597 bp, synthetic construct
  misc_feature 1..597: note = Clo51 E101N
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 94 - DNA, 597 bp, synthetic construct
  misc_feature 1..597: note = Clo51 E101A
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgcaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 95 - DNA, 597 bp, synthetic construct
  misc_feature 1..597: note = Clo51 E101L
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctctaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 96 - DNA, 597 bp, synthetic construct
  misc_feature 1..597: note = Clo51 E101I
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctataggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 97 - DNA, 597 bp, synthetic construct
  misc_feature 1..597: note = Clo51 E101G
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctggaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 98 - DNA, 597 bp, synthetic construct
  misc_feature 1..597: note = Clo51 E101T
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctacaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 99 - DNA, 597 bp, synthetic construct
  misc_feature 1..597: note = Clo51 E101F
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctttcggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 100 - DNA, 597 bp, synthetic construct
  misc_feature 1..597: note = Clo51 E101Y
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 101 - DNA, 597 bp, synthetic construct
  misc_feature 1..597: note = Clo51 E101W
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttggggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 102 - DNA, 597 bp, synthetic construct
  misc_feature 1..597: note = Clo51 E101P
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctccaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 103 - DNA, 597 bp, synthetic construct
  misc_feature 1..597: note = Clo51 E101H
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctcacggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 104 - DNA, 597 bp, synthetic construct
  misc_feature 1..597: note = Clo51 E101Q
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctcaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 105 - DNA, 597 bp, synthetic construct
  misc_feature 1..597: note = Clo51 E101R
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctagaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 106 - DNA, 597 bp, synthetic construct
  misc_feature 1..597: note = Clo51 E101M
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctatgggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 107 - DNA, 597 bp, synthetic construct
  misc_feature 1..597: note = Clo51 E101K
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctaaaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 108 - DNA, 597 bp, synthetic construct
  misc_feature 1..597: note = Clo51 E101V
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgtaggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 109 - DNA, 597 bp, synthetic construct
  misc_feature 1..597: note = Clo51 E101D
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactctgatggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 110 - DNA, 597 bp, synthetic construct
  misc_feature 1..597: note = Clo51 E101C
  source 1..597: mol_type = other DNA
gaaggtatcaagtccaacatctctttgttgaaggatgaattgaggggtcaaatctcccatatctctcatgaatacttgtccttgattgatttggccttcgactctaaacaaaacaggttgttcgaaatgaaggtcttggagttgttggttaacgaatacggttttaagggtagacacttaggtggttcaagaaaaccagatggtatcgtttactctaccaccttggaagataacttcggtattatcgttgataccaaggcttactcttgcggttactctttgccaatttctcaagctgacgaaatggaaagatacgtcagagaaaactccaacagagatgaagaggttaatccaaacaaatggtgggaaaacttctccgaagaagtcaagaagtactacttcgtctttatctccggttccttcaagggtaaattcgaagaacaattgaggcgtttgtctatgactactggtgttaatggttctgccgttaacgttgttaatttgttgttgggtgccgaaaagatcagatcaggtgaaatgactatcgaagaattggaaagagccatgttcaacaactctgagttcattttgaaatac

SEQ ID NO: 111 - DNA, 71 bp, synthetic construct
  misc_feature 1..71: note = SUF20 tRNA (Gly)
  source 1..71: mol_type = other DNA
gcgcaagtggtttagtggtaaaatccaacgttgccatcgttgggcccccggttcgattccgggcttgcgca

SEQ ID NO: 112 - protein, 1053 aa, synthetic construct
  REGION 1..1053: note = dSaCas9 (inactive small Cas9)
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 113 - protein, 5 aa, synthetic construct
  REGION 1..5: note = Linker
GGGGS

SEQ ID NO: 114 - protein, 1368 aa, synthetic construct
  REGION 1..1368: note = dCas9 without C-terminal NLS
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 115 - DNA, 4113 bp, synthetic construct
  misc_feature 1..4113: note = dCas9 without C-terminal NLS
  source 1..4113: mol_type = other DNA
atggacaaaaagtactctattggtttggccatcggtacaaattctgttggttgggctgttattactgatgagtacaaagttccctccaagaagtttaaggttttgggtaacactgacaggcactccattaagaaaaatttgattggtgccctgttgttcgattctggtgaaactgctgaagctactagattgaaaagaaccgctagaagaagatacaccagaagaaagaacagaatctgctacttgcaagagatcttctctaacgaaatggccaaggttgatgattcattcttccacagattggaagagtccttcttggttgaagaggataagaaacacgaaagacatccaatcttcggtaacatcgtagatgaagttgcttaccacgaaaagtacccaactatctaccatttgagaaagaagttggttgactctaccgataaggctgatttgagattgatctatttggctttggcccacatgattaagttcagaggtcatttcttgatcgagggtgatttgaatccagataactccgatgttgacaagttgttcatccaattggtccaaacttacaaccagctgtttgaagaaaatccaatcaatgcctctggtgttgatgctaaggctattttgtctgctagattgtccaagtctcgtaggttggaaaacttgattgctcaattgccaggtgaaaagaagaatggtttgttcggtaatttgatcgccttgtctttgggtttgactccaaatttcaagagcaacttcgatttggctgaagatgctaagttgcaattgtctaaggatacctacgatgacgatttggataacttgttggcacaaatcggtgatcaatacgctgatttgtttttggctgctaagaacttgtccgatgccattttgttgtccgatatcttgagagttaacaccgaaattactaaggctccattgtctgcctctatgatcaaaagatacgatgaacaccaccaagacttgaccttgttgaaagctttggttagacaacagttgcccgagaagtacaaagaaatcttcttcgaccaatccaagaacggttatgctggttatattgatggtggtgcttctcaagaagagttctacaagttcattaagcccatcttggaaaagatggatggtactgaagaactgttggtcaagttgaacagagaagatttgttgagaaagcagagaaccttcgataacggttctattccacatcaaatccacttgggtgaattgcatgctattttgagaaggcaagaggatttctacccattcttgaaggataacagagaaaagatcgagaagatcctgactttcagaatcccatattatgttggtccattggctagaggtaattccagatttgcttggatgactaggaagtctgaagaaactattactccatggaacttcgaagaggttgttgataagggtgcttctgctcaatccttcattgaaagaatgactaacttcgacaagaacctgccaaacgaaaaggttttgccaaagcactctctgttgtacgaatacttcactgtctacaacgaactgaccaaagttaagtacgttactgagggtatgagaaagccagcttttttgtctggtgaacaaaagaaggctatcgtcgacttgttgttcaagactaacagaaaggttaccgtcaagcaattgaaagaggactacttcaaaaagattgagtgcttcgattccgtcgaaatttcaggtgttgaagatagattcaacgcctccttaggtacttaccatgacttattgaagatcatcaaggataaggatttcttggacaacgaagaaaacgaggatatcttggaggatatcgttttgaccttgactttgttcgaagatagggaaatgatcgaggaaaggttgaaaacatacgcccatttgttcgatgacaaggtcatgaagcagttgaagagaagaaggtatactggttggggtagattgtcacgtaaattgattaacggtatcagggacaagcaatccggtaagactattttggacttcttgaagtctgatggtttcgccaatagaaacttcatgcaattgatccacgatgacagcttgaccttcaaagaagatattcaaaaggcccaagtttccggtcaaggtgattctttacatgaacatattgctaacttggctggttccccagctattaagaaaggtattttacaaaccgttaaggtggtcgacgaattggtaaaagttatgggtagacataagccagagaacatcgttattgaaatggctagagaaaatcagactacccaaaagggtcaaaagaactctagggaacgtatgaagagaattgaagagggtatcaaagagttgggttcccagattttgaaagaacacccagttgagaacactcaattgcagaacgaaaagctgtacttgtactacttgcagaatggtagagatatgtacgtcgaccaagagttggatatcaacagattgtctgattacgatgtcgatgctatcgttccacagtctttcttgaaagatgattccatcgacaacaaggtgttgaccagatctgataagaatagaggtaagtccgataacgtcccatctgaagaggtagttaagaagatgaagaattactggcgtcaactgttgaacgctaagttgattactcaaagaaagttcgacaacctgaccaaggctgaaagaggtggtttgtctgaattggataaggcaggtttcatcaagaggcaattggttgaaactagacaaatcactaagcacgttgcccaaatcttggatagtagaatgaataccaagtacgacgagaacgacaaattgatcagagaagttaaggtcatcacgctgaagtcaaagttggtttctgatttcagaaaggacttccagttttacaaggtcagggaaatcaacaactaccatcatgctcatgatgcttacttgaatgctgttgttggtactgccttgattaagaagtatccaaagttggaatccgagttcgtttacggtgattacaaggtttacgacgttagaaaaatgatcgccaagtccgaacaagaaattggtaaggctactgccaaatacttcttctactccaacatcatgaatttcttcaagaccgaaatcactttggccaacggtgaaattagaaaaaggccattgattgagactaacggtgagactggtgaaatagtttgggacaaaggtagagatttcgctaccgttagaaaggtcttgtctatgccacaagttaacatcgtcaaaaagaccgaagttcaaactggtggtttctccaaagagtctatcttgccaaaaagaaactccgataagttgatcgccagaaagaaagattgggacccaaaaaagtatggcggttttgattctccaactgttgcttactcagttttggttgttgctaaagttgaaaagggcaagagcaagaagttgaagtccgtcaaagaattattgggcatcacgattatggaaaggtcctcctttgaaaaaaaccctatcgattttttggaggccaagggttacaaagaggtcaaaaaggatctgattatcaagctgcctaagtactccttgttcgaactagaaaacggtagaaaaagaatgttggcttctgccggtgaattacaaaagggtaatgaattggctctgccatctaagtacgtcaactttctatacttggcctctcactacgaaaagttgaagggttctcctgaagataacgaacaaaagcagttgtttgtcgaacagcataagcactacttggacgaaatcattgaacagatctccgaattctccaagagggttattttggctgatgctaatttggacaaggttttgtccgcttacaacaagcacagagataagccaattagagaacaagccgaaaacatcattcacctgttcactttgactaatttgggtgctccagctgcttttaagtacttcgatacaaccatcgacagaaagagatacacctctaccaaagaagttttggacgctaccttgatccatcaatctattactggtctgtacgaaaccaggatcgacttgtctcaattaggtggtgatggatcctag

SEQ ID NO: 116 - protein, 7 aa, synthetic construct
  REGION 1..7: note = SV40 Nuclear Localization Sequence
PKKKRKV

SEQ ID NO: 117 - protein, 587 aa, unidentified
  REGION 1..587: note = NCBI Reference Sequence: ZP_05132802.1
  source 1..587: note = Clostridium sp. 7_2_43FAA
MINIIDVNNKTIRTFGWVQNPSNFESLKKVVAIFDNTSKTYNELKDKKIKKLVDERDGQKELLNALNANPLKIKYCNLVGTSFTPRSSARCNGIVQATVKGQRKEFIDDWSSDNFVRWAHALGFIKYNYDTDTFEITDVGRKYVQSEDDSNEESTILEEAMLSYPPVARVLTLLSNGEHLTKYEIGKKLGFVGEAGFTSLPLNVLIMTLATTDEPKEKNKIKTDWDGSSDKYARMISGWLVKLGLLVQRPKLVTVDFGGELYSETIGHAYMITDRGLKAVRRLLGINKVARVSKNVFWEMLATKGIDKNYIRTRRAYILKILIESNKVLTLEDIKGKLKLASINESINTIKDDINGLINTGINIKSETTGYKIYDSINDFIIPKTGDTEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 118 - protein, 208 aa, synthetic construct
  REGION 1..208: note = Nuclease domain of Clo051 with NLS without GS linker
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 119 - skipped

SEQ ID NO: 120 - protein, 5 aa, synthetic construct
  REGION 1..5: note = linker
GSGGS

SEQ ID NO: 121 - protein, 5 aa, synthetic construct
  REGION 1..5: note = linker
GGGGS

SEQ ID NO: 122 - protein, 7 aa, synthetic construct
  REGION 1..7: note = linker
GSGGGGS

SEQ ID NO: 123 - protein, 10 aa, synthetic construct
  REGION 1..10: note = linker
GGGGSGGGGS

SEQ ID NO: 124 - protein, 15 aa, synthetic construct
  REGION 1..15: note = linker
GGGGSGGGGSGGGGS

SEQ ID NO: 125 - protein, 7 aa, synthetic construct
  REGION 1..7: note = linker
GGGGSSA

SEQ ID NO: 126 - protein, 40 aa, synthetic construct
  REGION 1..40: note = linker
  VARIANT 3..40: note = up to 19 copies of GA can be deleted
GAGAGAGAGAGAGAGAGAGAGAGAGAGAGAGAGAGAGAGA

SEQ ID NO: 127 - protein, 60 aa, synthetic construct
  REGION 1..60: note = linker
  VARIANT 4..60: note = up to 19 copies of GGS can be deleted
GGSGGSGGSGGSGGSGGSGGSGGSGGSGGSGGSGGSGGSGGSGGSGGSGGSGGSGGSGGS

SEQ ID NO: 128 - protein, 100 aa, synthetic construct
  REGION 1..100: note = linker
  VARIANT 6..100: note = up to 19 copies of GGGGS can be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 129 - protein, 101 aa, synthetic construct
  REGION 1..101: note = linker
  VARIANT 2..101: note = up to 19 copies of GGGGS can be deleted
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 130 - protein, 40 aa, synthetic construct
  REGION 1..40: note = linker
  VARIANT 3..40: note = up to 19 copies of GS can be deleted
GSGSGSGSGSGSGSGSGSGSGSGSGSGSGSGSGSGSGSGS

SEQ ID NO: 131 - protein, 250 aa, synthetic construct
  REGION 1..250: note = linker
  REPEAT 1..5: note = single repeat of (GxSy)z where X is 1-4, Y is 0 or 1 and z is 1-50
  VARIANT 1..250: note = up to three 3 G residues and one S residue may can be deleted in each iteration of (G1-4 S0-1)
  VARIANT 6..250: note = up to 49 copies of (G1-4 S0-1) can be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 132 - protein, 100 aa, synthetic construct
  REGION 1..100: note = linker
  VARIANT 2..100: note = up to 99 G residues may be deleted
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 133 - protein, 5 aa, synthetic construct
  REGION 1..5: note = linker
EAAAK

SEQ ID NO: 134 - protein, 10 aa, synthetic construct
  REGION 1..10: note = linker
EAAAKEAAAK

SEQ ID NO: 135 - protein, 15 aa, synthetic construct
  REGION 1..15: note = linker
EAAAKEAAAKEAAAK

SEQ ID NO: 136 - protein, 20 aa, synthetic construct
  REGION 1..20: note = linker
EAAAKEAAAKEAAAKEAAAK

SEQ ID NO: 137 - protein, 25 aa, synthetic construct
  REGION 1..25: note = linker
EAAAKEAAAKEAAAKEAAAKEAAAK

SEQ ID NO: 138 - protein, 7 aa, synthetic construct
  REGION 1..7: note = linker
GSGGGGS

SEQ ID NO: 139 - protein, 7 aa, synthetic construct
  REGION 1..7: note = linker
GGGGSSA

SEQ ID NO: 140 - protein, 13 aa, synthetic construct
  REGION 1..13: note = linker
GGGGSGGGGSAAA